(19) United States
(12) Patent Application Publication
(10) Pub. No.: US 2024/0415545 A1
(43) Pub. Date: Dec. 19, 2024

(54) STABILIZATION IMPLANT SYSTEMS AND RELATED METHODS

(71) Applicant: The Orthopaedic Implant Company, Reno, NV (US)

(72) Inventor: Petr COLWELL, Reno, NV (US)

(73) Assignee: The Orthopaedic Implant Company, Reno, NV (US)

(21) Appl. No.: 18/336,012

(22) Filed: Jun. 15, 2023

(51) Int. Cl. A61B 17/68; A61B 17/84

(52) U.S. Cl. CPC A61B 17/683; A61B 17/842; A61B 2017/00367; A61B 2017/0404; A61B 2017/0409; A61B 2017/0414; A61B 2017/564

(57) ABSTRACT

Implant systems for stabilizing first and second bones comprising an implant and an implantation instrument. The implant comprises first and second anchors coupled by a flexible tether. The instrument comprises a handle, a manually engageable adjustment member, a longitudinally elongated insertion tube extending from the handle to a tip comprising a longitudinally elongated cavity, and a deployment portion. The deployment portion is threadably engaged with the instrument, and comprises a base portion extending within the handle and a longitudinally elongated deployment rod portion slidably received within the cavity. The first bone anchor is removably received within the cavity at the tip thereof. Movement of the adjustment member in a first direction adjusts the threaded engagement of the deployment portion with respect to the instrument to threadably drive the deployment rod portion longitudinally within the cavity toward the tip thereof to force the first bone anchor out from the cavity.

### TECHNICAL FIELD

The present disclosure relates generally to orthopedic surgery related to dynamic stabilization of bones and/or tissue. More specifically, but not exclusively, the present disclosure relates to implant devices, instruments, systems, and methods that achieve stabilization of a first and second bones or tissues.

### BACKGROUND

A syndesmosis joint is a fibrous joint where two bones are connected by strong ligaments or membrane. The distal tibiofibular syndesmosis, between the fibula and tibia, is formed by three major ligaments: the anterior inferior tibiofibular ligament (AITFL), the posterior inferior tibiofibular ligament (PITFL), and the interosseous tibiofibular ligament (ITFL). A fourth ligament, the inferior transverse tibiofibular ligament, is congruent with the PITFL, but sometimes considered a separate ligament. While technically the syndesmosis is the joint, most literature describes a syndesmosis injury as affecting the syndesmotic ligaments.

Syndesmotic injuries most typically occur to the distal tibiofibular syndesmosis, and result from trauma (such as, but not limited to, sports injuries). Distal tibiofibular syndesmotic injuries can occur when the stabilizing tibiofibular ligaments are damaged during extreme ankle roll/rotation. Forceful external rotation and/or abduction of an ankle can widen the ankle mortise, as the talus pushes the distal fibula laterally away from its articulation with the distal tibia. Stretching and/or tearing of the syndesmosis, deltoid and associated ligamentous structures can result in severe pain, ankle instability and/or diastasis. A distal fibular fractur usually above the joint line, if often involved with more severe injuries. Syndesmotic injuries can occur as a purely ligamentous injury, or in combination with a bone fracture, where the syndesmotic ligaments may become disrupted, separated, or injured.

The current standard of care for syndesmotic injuries typically involves rigid fixation with one or more screws. Rigid screw-based fixation is simple to implant and stabilizes the syndesmotic joint, but fails to allow any motion at all, as would normally exist physiologically. This limits the patient's range of motion, and unpredictable screw failure locations can result in damage to existing bone and/or patient pain.

Currently commercially available suture/tethered-based syndesmosis stabilization/constraint implant systems allow for limited motion of the joint, but include implantation disadvantageous. For example, many such suture-based syndesmosis stabilization implant systems are configured to be implanted via a medial side of the joint through a medial incision, which is anatomically disadvantageous for the patient. Further, current suture-based syndesmosis stabilization implant systems do not provide feedback during deployment, and thereby can result in implantation errors and/or difficulties.

Other bones and tissues, such as but not limited to other joints, suffer from similar issues as syndesmotic joints. Further, commercially available suture/tethered-based stabilization/constraint implant systems for such other bones and tissues suffer from the same or similar drawbacks as syndesmosis stabilization/constraint implant systems.

Thus, new and improved stabilization devices, instruments, systems and methods are needed to overcome drawbacks of the currently available solutions.

### SUMMARY

The present disclosure is directed toward syndesmotic implants, systems and methods for use in dynamic stabilization of a syndesmotic joint (i.e., constrained and controlled movement and/or forces in the joint). While the implants, systems and methods disclosed herein (and described in detail below) are particularly advantageous for use in, and dynamic stabilization of, distal tibiofibular syndesmosis, the implants and methods may be configured and utilized for use with any joint, and in particular any syndesmotic joint. The stabilization syndesmotic implants, systems and methods provide for dynamic stabilization of a syndesmotic joint, and advantageous implantation techniques and constructs. For example, with respect to dynamic stabilization of the distal tibiofibular syndesmosis, the dynamic stabilization syndesmotic implants, systems and methods are advantageously configured to provide feedback of the implantation of a medial anchor member, and avoid the need for a medial incision to implant the medial anchor member. The dynamic stabilization syndesmotic implants, systems and methods of the present disclosure thus advantageously avoid medial incisions, and thereby damage to medial tissue and other anatomical structures, by only necessitating a lateral incision/opening. Further, the dynamic stabilization syndesmotic implants, systems and methods of the present disclosure also advantageously provide tangible feedback and controlled incremental/gradual/slow implantation/deployment to ensure accurate implantation that avoid damage or irritation to nearby anatomical structures.

In one aspect, the present disclosure provides an implant system for stabilizing first and second bones comprising a stabilization implant and an instrument configured to deploy the implant across a portion of the first and second bones. The stabilization implant comprises a first bone anchor, a second bone anchor and a flexible tether extending between and coupling the first and second anchors. The instrument comprises a handle portion with a manually engageable adjustment member movably coupled thereto, and a longitudinally elongated insertion tube extending from a front portion of the handle portion to a tip and comprising a longitudinally elongated cavity. The instrument further comprises a deployment portion coupled with the adjustment member and threadably engaged with a portion of the instrument, the deployment portion comprising a base portion extending within the handle portion and a longitudinally elongated deployment rod portion slidably received within the cavity of the insertion tube. At least a portion of the first bone anchor is removably received within the cavity of the insertion tube at the tip thereof. Movement of the adjustment member in a first direction adjusts the threaded engagement of the deployment portion with respect to the instrument to threadably drive the deployment rod portion longitudinally within the cavity of the insertion tube toward the tip thereof to force the first bone anchor out from the cavity.

In some embodiments, the deployment portion is threadably engaged with the portion of the instrument via the base portion being threadably engaged with an inner portion of the handle portion. In some such embodiments, the base portion comprises external threads, and the inner portion of the handle portion comprises interior threads threadably mated with the external threads. In some other such embodiments, the base portion is rotatably and longitudinally slidably received with the handle portion, and wherein the adjustment member is rotationally fixedly coupled with the base portion. In some such embodiments, the first direction is a rotational direction extending about a longitudinal axis, and wherein rotation of the adjustment member in the first direction rotates the base portion within the handle portion and threadably drives the base portion and the rod portion longitudinally toward the tip. In some such embodiments, the adjustment member is configured as a thumb wheel that extends within the handle portion and exterior to an outer surface of the handle portion. In some other such embodiments, the base portion is longitudinally slidably coupled with the adjustment member.

In some embodiments, the base portion of the deployment portion extends through a back end of the handle portion such that a back end portion of the base portion is positioned past the back end of the handle portion, and wherein the instrument further comprises a safety clip removably coupled with the back end portion of the base member that engages with the back end of the handle portion to prevent longitudinal movement of the deployment rod portion within the cavity of the insertion tube toward the tip thereof. In some embodiments, the base portion of the deployment portion extends through an opening in a back end of the handle portion such that a back end portion of the base portion is positioned past the back end of the handle portion, and wherein at least a first portion of the back end portion of the base portion defines a dimension larger than the opening such that longitudinal movement of the deployment portion toward the tip is prevented when the first portion engages the back end of the handle portion.

In some embodiments, the second bone anchor member is removably retained within a retention opening of an anchor retention portion of the handle portion. In some such embodiments, the e tether is in longitudinal tension between the first and second bone anchors to selectively retain the first bone anchor within the cavity of the insertion tube and the second bone anchor within the retention opening of the anchor retention portion until after deployment of the implant.

In some embodiments, the first bone anchor comprises a first pair of longingly spaced tether apertures each extending through a thickness thereof between an engagement side and an outer side thereof, and wherein an intermediate portion of the tether extends through a first tether aperture of the first pair of tether apertures from the engagement side to the outer side, and then through a second tether aperture of the first pair of tether apertures from the outer side to the engagement side. In some such embodiments, the second bone anchor comprises a second pair of tether apertures, and wherein a first end portion of the tether extends through a third tether aperture of the second pair of tether apertures, a second end portion of the tether extends through a fourth tether aperture of the second pair of tether apertures, and the intermediate portion of the tether extends between the first and second end portions of the tether. In some such embodiments, the second bone anchor comprises a head portion and a shaft portion extending from the head portion, wherein the second pair of tether apertures are formed by a longitudinal opening in the head portion and a lateral through hole through the shaft portion of the second anchor that is in communication with the longitudinal opening. In some such embodiments, the first end portion of the tether extends through the first tether aperture from the head portion to the shaft portion, and the second end portion of the tether extends through the second tether aperture from the shaft portion to the head portion. In some embodiments, the first and second end portions of the tether are arranged in a slip knot that is configured to retain the length of the intermediate portion of the tether extending between the first and second anchors.

In some embodiments, the first bone anchor is longitudinally elongated, and wherein the first bone anchor is longitudinally oriented with respect to the longitudinally elongated cavity of the insertion tube, and at least a portion of the first bone anchor along the longitudinal length thereof is removably received within a portion of the longitudinally elongated cavity of the insertion tube at the tip thereof.

In some embodiments, the instrument further comprises a tether retainer comprising a pair of projections with wing portions and slot between the wing portions, and wherein end portions of the tether wrap about the projections beneath the wing portions and pass through the slot at least once. In some such embodiments, the instrument further comprises a tether retainer keeper comprising an elastic member with an opening, the tether retainer keeper being configured to extend about the projections beneath the wing portions and over the end portions of the tether wrapped thereabout in a resiliently deformed expanded state.

In some embodiments, the second bone anchor comprises a head portion and a shaft portion extending from the head portion, wherein the implant further comprises a washer member with a through hole, the washer member being configured to engage the head portion of the second bone anchor with the shaft portion of the second bone anchor extending through the through hole, and wherein the washer member is mounted on the instrument with the insertion tube and the tether extending through the through hole.

In another aspect, the present disclosure provides a method of stabilizing first and second bones. The method comprises obtaining an implant system comprising a stabilization implant mounted on an implantation instrument. The stabilization implant comprises first and second bone anchors and a flexible tether coupling the first and second bone anchors. The implantation instrument comprises a handle portion, an adjustment member, a longitudinally elongated insertion tube extending from the handle portion to a tip and comprising a longitudinally elongated cavity, and a deployment portion coupled with the adjustment member and threadably engaged with a portion of the instrument. The deployment portion comprises a base portion extending within the handle portion and a longitudinally elongated deployment rod portion slidably received within the cavity of the insertion tube. At least a portion of the first bone is removably received within the cavity of the insertion tube at the tip thereof. The method further comprises passing the first bone anchor and a portion of the insertion tube from an outer side of the first bone through a portion of the first and second bones such that the second bone anchor is positioned past an outer side of the first bone and the first bone anchor is positioned adjacent to an outer side of the second bone. The method also comprises manually adjusting the adjustment member in a first direction to threadably drive the deployment rod portion longitudinally within the cavity of the insertion tube toward the tip thereof to force the first bone anchor out from the cavity. The method further comprises adjusting the location of the second bone anchor along the tether to force to shorten a length of the tether between the first and second bone anchors to position the second bone anchor against the outer side of the first bone. The method also comprises tying end portions of the tether extending from the second bone anchor into a surgical knot to fix the arrangement of the implant and stabilize the first and second bones.

In some embodiments, the first and second bones are bones of a syndesmosis joint.

In another aspect, the present disclosure provides an instrument for implanting an implant through first and second bones comprising a handle portion, a longitudinally elongated insertion tube and a deployment portion. The handle portion comprises a manually engageable adjustment member movably coupled thereto. The longitudinally elongated insertion tube extends from a front portion of the handle portion to a tip and comprises a longitudinally elongated cavity, the cavity at the tip being configured to removably house at least a portion of a first bone anchor of the implant. The deployment portion is coupled with the adjustment member and threadably engaged with a portion of the instrument, and comprises a base portion extending within the handle portion and a longitudinally elongated deployment rod portion slidably received within the cavity of the insertion tube, Movement of the adjustment member in a first direction adjusts the threaded engagement of the deployment portion with respect to the instrument to threadably drive the deployment rod portion longitudinally within the cavity of the insertion tube toward the tip thereof.

In some embodiments, the deployment portion is threadably engaged with the portion of the instrument via the base portion being threadably engaged with an inner portion of the handle portion. In some embodiments, the handle portion further comprises a bone anchor retention portion configured to removably retain a second bone anchor of the implant that is coupled to the first bone anchor via a flexible tether.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating embodiments of the inventions, and are not to be construed as limiting the disclosure.

FIG. 1 illustrates a perspective side view of an exemplary joint stabilization implant system, in accordance with an aspect of the present disclosure;

FIG. 2 illustrates a perspective side view of a first anchor and a tether of the implant system of FIG. 1, in accordance with an aspect of the present disclosure;

FIG. 3 illustrates a perspective side view of a second anchor and the tether of the implant system of FIG. 1, in accordance with an aspect of the present disclosure;

FIG. 4 illustrates a side cross-sectional view of the first and second anchor and the tether of the implant system of FIG. 1, in accordance with an aspect of the present disclosure;

FIG. 5 illustrates a top perspective view of the first anchor and the tether of the implant system of FIG. 1 implanted into a first bone, in accordance with an aspect of the present disclosure;

FIG. 6 illustrates a top perspective view of the second anchor and the tether of the implant system of FIG. 1 implanted into a second bone, in accordance with an aspect of the present disclosure;

FIG. 7 illustrates a top perspective view of the first anchor of the implant system of FIG. 1 implanted into a first bone, in accordance with an aspect of the present disclosure;

FIG. 8 illustrates a bottom perspective view of the first anchor of the implant system of FIG. 1 implanted into a first bone, in accordance with an aspect of the present disclosure;

FIG. 9 illustrates a top/bottom view of the first anchor of the implant system of FIG. 1 implanted into a first bone, in accordance with an aspect of the present disclosure;

FIG. 10 illustrates a side view of the first anchor of the implant system of FIG. 1 implanted into a first bone, in accordance with an aspect of the present disclosure;

FIG. 11 illustrates an end view of the first anchor of the implant system of FIG. 1 implanted into a first bone, in accordance with an aspect of the present disclosure;

FIG. 12 illustrates a top perspective view of the second anchor of the implant system of FIG. 1 implanted into a first bone, in accordance with an aspect of the present disclosure;

FIG. 13 illustrates another top perspective view of the second anchor of the implant system of FIG. 1 implanted into a first bone, in accordance with an aspect of the present disclosure;

FIG. 14 illustrates a bottom perspective view of the second anchor of the implant system of FIG. 1 implanted into a first bone, in accordance with an aspect of the present disclosure;

FIG. 15 illustrates a side view of the second anchor of the implant system of FIG. 1 implanted into a first bone, in accordance with an aspect of the present disclosure;

FIG. 16 illustrates a top view of the second anchor of the implant system of FIG. 1 implanted into a first bone, in accordance with an aspect of the present disclosure;

FIG. 17 illustrates a bottom view of the second anchor of the implant system of FIG. 1 implanted into a first bone, in accordance with an aspect of the present disclosure;

FIG. 18 illustrates a top view of a washer member configured for use with the second anchor of the implant system of FIG. 1, in accordance with an aspect of the present disclosure;

FIG. 19 illustrates a side view of the washer member of FIG. 18, in accordance with an aspect of the present disclosure;

FIG. 20 illustrates a top perspective view of the first anchor of the implant system of FIG. 1 and the washer member of FIG. 18 implanted into a first bone, in accordance with an aspect of the present disclosure;

FIG. 21 illustrates a top perspective view of the first anchor of the implant system of FIG. 1 and a bone plate implanted into a first bone, in accordance with an aspect of the present disclosure;

FIG. 22 illustrates a top front perspective view of an implantation instrument with the implant system of FIG. 1 loaded thereon, in accordance with an aspect of the present disclosure;

FIG. 23 illustrates a top rear perspective view of the implantation instrument of FIG. 22 with a tether retainer thereof removed, in accordance with an aspect of the present disclosure;

FIG. 24 illustrates a top view of the implantation instrument of FIG. 22, in accordance with an aspect of the present disclosure;

FIG. 25 illustrates a side cross-sectional view of a portion of the implantation instrument of FIG. 22, in accordance with an aspect of the present disclosure;

FIG. 26 illustrates a side cross-sectional view of another portion of the implantation instrument of FIG. 22, in accordance with an aspect of the present disclosure;

FIG. 27 illustrates a top rear perspective view of a tether retainer portion of the implantation instrument of FIG. 22 with a tether retainer keeper removed, in accordance with an aspect of the present disclosure;

FIG. 28 illustrates a top rear perspective view of a tether retainer portion and a tether retainer keeper of the implantation instrument of FIG. 22, in accordance with an aspect of the present disclosure;

FIG. 29 illustrates a side view of the implantation instrument of FIG. 22 with the second anchor member decoupled from an anchor retainer portion of the instrument, in accordance with an aspect of the present disclosure;

FIG. 30 illustrates a top view of the implantation instrument of FIG. 22 with the second anchor member being coupled with an anchor retainer portion of the instrument, in accordance with an aspect of the present disclosure;

FIG. 31 illustrates a top rear perspective view of the implantation instrument of FIG. 22 without the implant system and a tether retainer portion and a safety clip of the instrument decoupled from the instrument, in accordance with an aspect of the present disclosure;

FIG. 32 illustrates a top front exploded perspective view of the implantation instrument of FIG. 31, in accordance with an aspect of the present disclosure;

FIG. 33 illustrates a top front partial-exploded perspective view of the implantation instrument of FIG. 31, in accordance with an aspect of the present disclosure;

FIG. 34 illustrates a top side exploded perspective view of the implantation instrument of FIG. 31, in accordance with an aspect of the present disclosure;

FIG. 35 illustrates a top side exploded perspective view of the implantation instrument of FIG. 31 without a portion of a housing of the instrument, in accordance with an aspect of the present disclosure;

FIG. 36 illustrates a side view of the implantation instrument of FIG. 31 without a portion of the housing of the instrument, in accordance with an aspect of the present disclosure;

FIG. 37 illustrates an elevated rear side perspective view of the implantation instrument of FIG. 31 without a portion of the housing of the instrument, in accordance with an aspect of the present disclosure;

FIG. 38 illustrates an elevated rear side exploded perspective view of the implantation instrument of FIG. 31 without a portion of the housing of the instrument, in accordance with an aspect of the present disclosure;

FIG. 39 illustrates a side view of a deployment mechanism portion of the implantation instrument of FIG. 31, in accordance with an aspect of the present disclosure;

FIG. 40 illustrates a side view of a safety clip portion of the implantation instrument of FIG. 31, in accordance with an aspect of the present disclosure;

FIG. 41 illustrates an elevated side perspective view of the implantation instrument of FIG. 22, with a washer member mounted on the implant, inserting the implant into first and second bones, in accordance with an aspect of the present disclosure;

FIG. 42 illustrates an elevated side perspective view of the implantation instrument and implant of FIG. 41 deploying the implant into the first and second bones, in accordance with an aspect of the present disclosure;

FIG. 43 illustrates an elevated side perspective view of the implantation instrument of FIG. 22 illustrating movement of the instrument and first anchor during deployment of the implant into the first and second bones, in accordance with an aspect of the present disclosure;

FIG. 44 illustrates movement of the first anchor during deployment of the first anchor into the second bone during the implant deployment of FIG. 42, in accordance with an aspect of the present disclosure;

FIG. 45 illustrates an elevated side perspective view of the implantation instrument of FIG. 22 inserting the implant into first and second bones with a bone plate coupled to the first bone, in accordance with an aspect of the present disclosure; and

FIG. 46 illustrates an elevated side perspective view of the implantation instrument and implant of FIG. 45 deploying the implant into the first and second bones and the one plate, in accordance with an aspect of the present disclosure.

### DETAILED DESCRIPTION

Generally stated, disclosed herein are devices and systems for achieving bone stabilization. Further, methods for using the devices and systems to achieve ligament fixation are discussed.

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

Similarly, positions or directions may be used herein with reference to anatomical structures or surfaces. For example, as the current implants, devices, instrumentation and methods are described herein with reference to use with the bones of the ankle/leg, the bones of the foot, ankle and lower leg may be used to describe the surfaces, positions, directions or orientations of the implants, devices, instrumentation and methods. Further, the implants, devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the disclosure. For example, the implants, devices, instrumentation and methods, and the aspects, components, features and the like thereof, described herein with respect to the right leg may be mirrored so that they likewise function with the left leg. Further, the implants, devices, instrumentation and methods, and the aspects, components, features and the like thereof, disclosed herein are described with respect to the leg for brevity purposes, but it should be understood that the implants, devices, instrumentation and methods may be used with other bones of the body having similar structures.

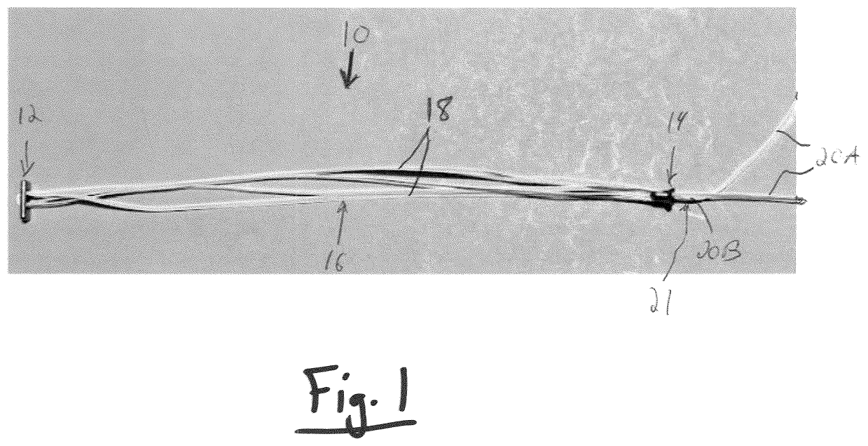

Referring to the drawings, wherein like reference numerals are used to indicate like or analogous components throughout the several views, and with particular reference to FIGS. 1-21 there is illustrated an exemplary embodiment of an implant device or construct (or system) 10 according to the present disclosure. The implant device/construct/system 10 may be, for example, configured to heal ligaments (e.g., syndesmotic ligaments) post-operatively and/or stabilize two bones (separate and distinct bones or bone segments), such as a joint between two bones (e.g., a syndesmotic joint). In one exemplary embodiment, the implant 10 is advantageously configured to being implanted across a syndesmotic joint, such as but not limited to a distal tibiofibular syndesmosis, to stabilize the syndesmotic joint. The implant device/construct 10 is configured to selectively constrain motion between two or more bones or tissues (e.g., in all directions), such as to allow healing therebetween to stabilize the relationship therebetween. For example, in one embodiment, the implant device/construct 10 is configured to selectively constrain motion between two or more syndesmotic bones to allow healing of one or more syndesmotic ligaments extending therebetween to heal and/or to stabilize the syndesmotic joint. The implant device/construct 10 allows for only a limited amount of relative movement/motion between bones/tissue in which first and second anchors thereof are implanted or installed, and such relative movement/motion and/or forces acting between the bones/tissue is controlled movement/motion (e.g., resisted/inhibited and/or encouraged/enhanced).

The components and portions of the dynamic implant device/construct 10 may be made of, for example, titanium, stainless steel, polymers, polyester, polypropylene, UHMWPE, thermoplastic (e.g., thermoplastic urethane), bio-resorbable materials or any other biocompatible material so that the implant device/construct 10 is configured to be implanted into a mammalian patient, such as a human patient. Some components of the dynamic implant device/construct 10, such as bone or tissue anchors as explained further below, may be formed as stiff and strong components that resist bending/flexing or other deformation during use, and other components of the dynamic implant device/construct 10, such as at least one suture or tether that extends between the anchors as explained further below, may be formed as a freely flexible and conformable component that that bends and otherwise changes shape during use (e.g., to provide the dynamic nature of the construct).

As noted above, the implant device/construct 10 provides stabilization/fixation of a joint or space between two bones (either naturally distinct bones or bone portions/segments), such as bones of a syndesmotic joint (e.g., a distal fibula and tibia of a distal tibiofibular syndesmosis). The implant device/construct 10 is configured to provide stabilization through constrained and/or controlled relative motion between the bones. The area of allowed constrained controlled motion provided by the implant device/construct 10 may be set in a space or gap between adjacent bones (such as the syndesmotic joint/space between the distal the fibula and tibia). In some embodiments, the implant device/construct 10 may be utilized, and may be particularly configured for, use in bones or joints other than a syndesmotic joint and/or with soft tissue as opposed to two bones.

As shown in FIGS. 1-6, 20 and 21, the stabilization implant device/construct/system 10 includes a flexible suture or tether 16 as a constraint and/or tension member that extends through portions of (or is otherwise coupled with), and from/between, a first bone and/or tissue anchor 12 and a second bone and/or tissue anchor 14. The tether 16 thereby connects and extends between the first and second anchors 12, 14 to form the implant device or construct 10. In some embodiments, the stabilization implant 10 includes at least the first and second anchors 12, 14 and the tether 16 (i.e., includes at least the three components). In some embodiments, the stabilization implant 10 only includes the first and second anchors 12, 14 and the tether 16 (i.e., is a three-piece or three-component implant that consists of only the first and second anchors 12, 14 and the tether 16). The first anchor 12 and the second anchor 14 are configured to respectively be coupled to, and may or may not be implanted at least partially within, first and second bones or tissues, such as bones of a syndesmotic joint (e.g., a distal fibula and tibia, respectively, of a distal tibiofibular syndesmosis), with the tether 16 extending between and coupled to the first anchor 12 and the second anchor 14. It is noted that the first and second bones may be separate and distinct bones (e.g., a fibula and a tibia), or bone segments or portions (e.g., portions of a bone, such as portions being separated by a fracture defect, disease or the like). Similarly, the first anchor 12 and/or the second anchor 14 may be configured to respectively coupled to, and be implanted at least partially within, tissue that is not bone tissue, such as a soft tissue (ligament, tendon, cartilage, muscle, etc.). In some embodiments, the first anchor 12 and/or the second anchor 14 may be formed of a rigid biocompatible material, such as but not limited to a biocompatible metal material (e.g., stainless steel, cobalt or a cobalt-based alloy, titanium or a titanium-based allow (Ti-6Al-4V), etc.), a biocompatible plastic or synthetic material, a biocompatible ceramic material, etc.). In some embodiments, the first anchor 12 and/or the second anchor 14 may be of one-piece construction and/or monolithic.

As noted above, the implant 10 may be particularly advantageous as a distal tibiofibular syndesmosis stabilization implant device/construct 10. In such an embodiment, the first anchor 12 may be configured as a medial anchor (e.g., a medial bone anchor) for positioning and engagement with the medial side of a distal tibia of a distal tibiofibular syndesmosis (e.g., a medial tibia cortex), and the second anchor 14 may be configured as a lateral anchor (e.g., a lateral bone anchor) for positioning and engagement with the lateral side of a distal fibula of the distal tibiofibular syndesmosis (e.g., slightly above the joint line and slightly posterior of the AP (anterior-posterior) plane of the fibula), as described further below with respect to FIGS. 41-46. In some embodiments, the first bone anchor 12 may be configured to be implanted from a first outer side of a first bone through a portion of the first bone (while the second bone anchor 14 is positioned generally at or in abutment with the outer side of the first bone), and through an adjacent or nearby second bone from a first outer side to a second outer side thereof, such that the first bone anchor is positioned past the second outer side of the second bone and the second bone anchor is (remains) positioned adjacent to the first outer side of the first bone. The implant 10 may thereby be implanted from the second outer side of the second bone (via the first bone anchor 12) to the first outer side of the first bone (via the second bone anchor 14), and extend thereby between (via the tether 16) to stabilize (and potentially bias or compress together) the first and second bones with respect to each other. In one exemplary embodiment, the second outer side of the second bone is at (or comprises) a medial side of the second bone, and the first outer side of the first bone is at (or comprises) a lateral side of the first bone. However, it is specifically noted that the second outer side of the second bone and the first outer side of the first bone may be any sides or portions of the respective bones.

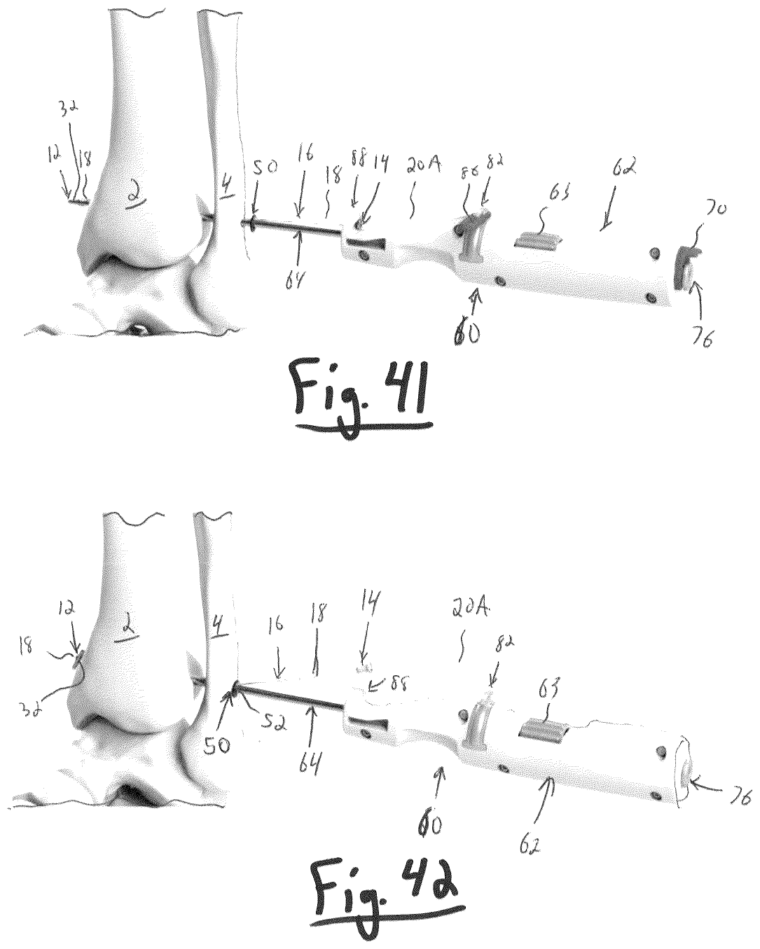

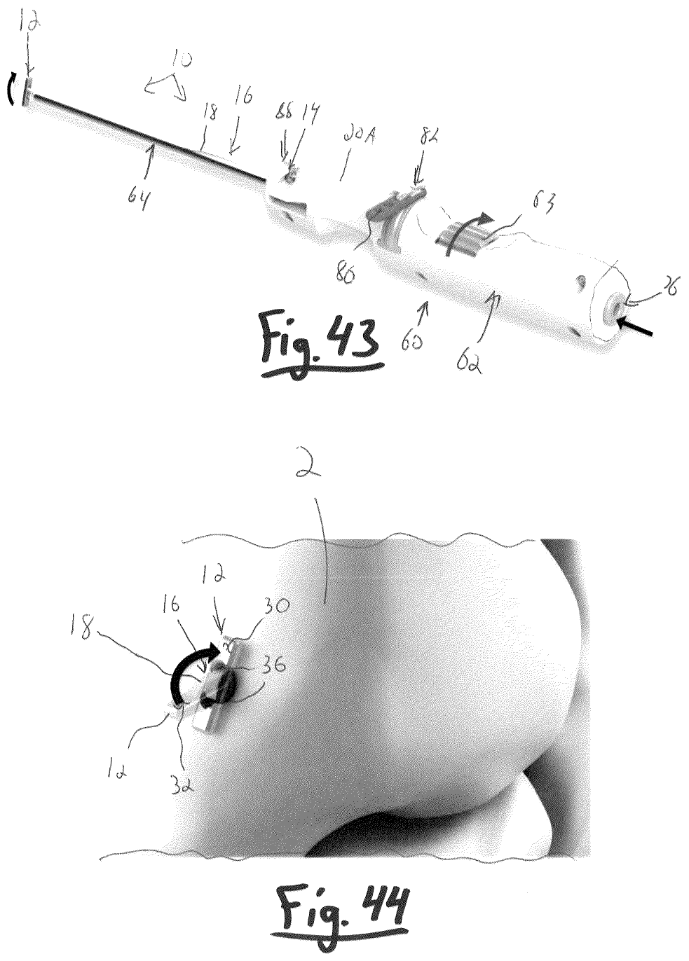

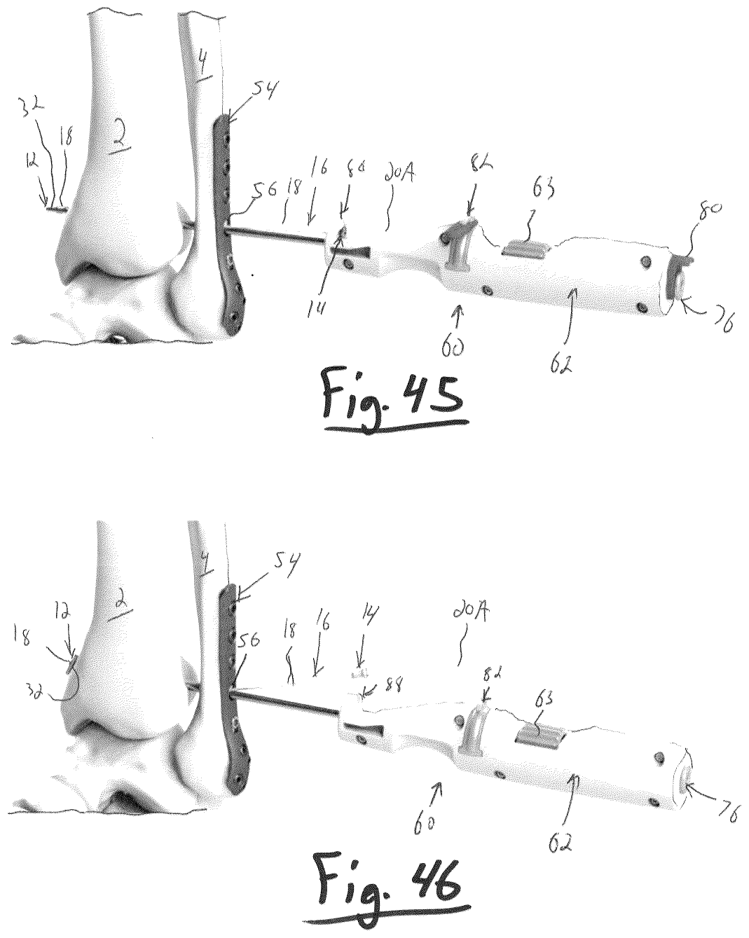

As shown in FIGS. 1-6 and 41-46, the tether 16 may be configured to mimic the function, location, strength and/or length of a ligament, for example but not limited to a syndesmotic ligament (e.g., an interosseous ligament.) The tether 16 may be of any thin, elongate and substantially freely manually flexible configuration or structure, such as a suture, strand, cable or string-like configuration. The tether 16 may be, for example, a biomedical suture or tether (e.g., a stranded cerclage cable), or similar construct. In some embodiments, the tether 16 may be formed of, for example, polymer, polyester, polypropylene or UHMWPE/HMWPE suture, tape, strands or filaments, braids thereof, or a like material/configuration, as known by one of ordinary skill in the art. The tether 16 may be, for example, a suture, such as a single cross-section suture tape or multiple loops of suture tape (e.g., a UHMWPE/HMWPE suture tape). For example, in the exemplary illustrated embodiment of the implant system/construct 10, the tether 16 is a single piece/length of suture tape (e.g., woven UHMWPE/HMWPE suture tape, about 1 mm suture tape, about 30 long), which may be doubled over upon itself such that a first free end portion 20A of the tether 16 comprises/is formed by a pair of separate and distinct free ends of the tether 16 and the other second free end portion 20B of the tether 16 comprises/is formed by an end loop or looped end of the tether 16, and an intermediate loop portion 18 extends between the first and second end portions 20A, 20B, as shown in FIGS. 1 and 4. The tether 16 may thereby form a double strand or double tape loop arrangement or configuration as it extends through and between the first and second anchors 12, 14, as shown in FIGS. 1-4.

In an initial, first or adjustment state, arrangement or configuration of the implant 10, as shown in FIGS. 1-4, the first anchor 12 may be slidably coupled with an intermediate loop portion 18 of the tether 16 extending from the first anchor 12, and the second anchor 14 may be slidably coupled with the end portions 20A, 20B of the tether 16. In the adjustment state, the tether 16 and the first and second anchors 12, 14 are configured such that the second end portions 20B can be tensioned and pulled/tensioned, and portions of the intermediate portion 18 pulled/translated through the second anchor 14 such that the intermediate loop portion 18 slides through the first anchor 12 and is shortened, to thereby draw the first and second anchors 12, 14 closer together (i.e., the intermediate loop portion 18 extending between the first and second anchors 12, 14 is shortened).

In the adjustment state, the first and second end portions 20A, 20B may be arranged, configured or tied into a one-way adjustment knot 21, such as a slip knot, positioned past a bearing portion 48 of the second anchor 14 (i.e., on an opposing side of the portion of the second anchor 14 than the intermediate portion 18), as shown in FIGS. 1, 3 and 4. The adjustment knot 21 is configured to allow portions of the intermediate portion 18 to be pulled/translated through the adjustment knot 21 (and the second anchor 14) via tension applied to the second end portions 20B away from a head portion 40 of the second anchor 14 (and the intermediate portion 18) to shorten the intermediate portion 18, and thereby draw the first and second anchors 12, 14 together. The adjustment knot 21 is further configured to prevent or resist the first and second end portions 20A, 20B from being pulled/translated through the adjustment knot 21 (and thereby the second anchor 14) via tension applied to the intermediate portion 18 away from a stem portion 42 of the second anchor 14 toward the first anchor 12 (and the first and second end portions 20A, 20B) to enlarge the intermediate portion 18, and thereby allow the first and second anchors 12, 14 to translate away from each other. The adjustment knot 21 may be configured such that the adjustment knot 21 tightens (upon itself and/or the bearing portion 48) when the intermediate portion 18 (and thus the first and second end portions 20A, 20B) are tensioned in a direction extending between the first and second end portions 20A, 20B. The adjustment knot 21 and the second anchor 14 are also configured to prevent the first and second end portions 20A, 20B (and the adjustment knot 21 itself) from passing through the second anchor 14 toward the first anchor 12 (as it cannot pass over/past the bearing portion 48 of the second anchor 14), as explained further below. The adjustment knot 21 is thereby configured to allow a user to selectively shorten the intermediate portion 18 (via tension/translation of the second end portions 20B), and prevent or resist elongation or enlargement of the intermediate portion 18, and prevent the first and second end portions 20A, 20B from passing through the second anchor 14 (in a direction extending from the head portion 40 to the stem portion 42 thereof).

The adjustment knot 21 may be any configuration or arrangement of the first and second end portions 20A, 20B of the tether 16 that is effective in providing the one-way adjustability described above. In some embodiments, the adjustment knot 21 is a sliding or slip knot. In some embodiments, the adjustment knot 21 is formed via at least two half hitch or similar knots or configuration, such as formed via the second end portion 20B, as shown in FIG. 4. As noted above, the second end portion 20B of the tether 16 may comprise a loop end. As shown in FIG. 4, in some embodiments the adjustment knot 21 may be formed via the first end portions 20A extending through at least one self-tightening hitch type knot or looped arrangement of the second end portion 20B of the tether 16.

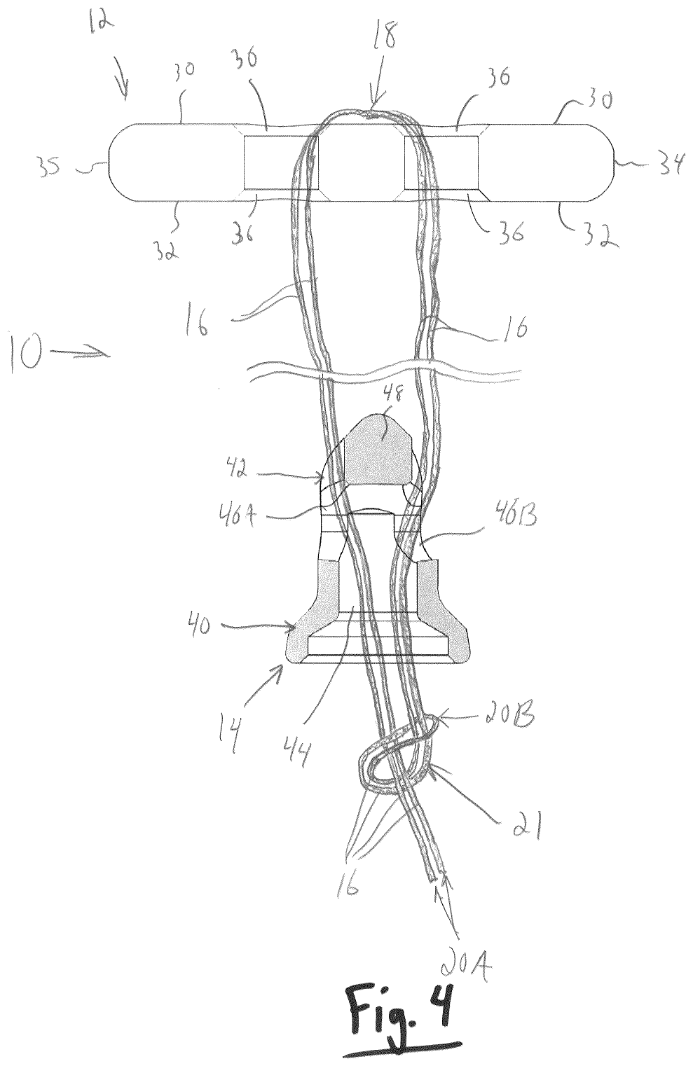

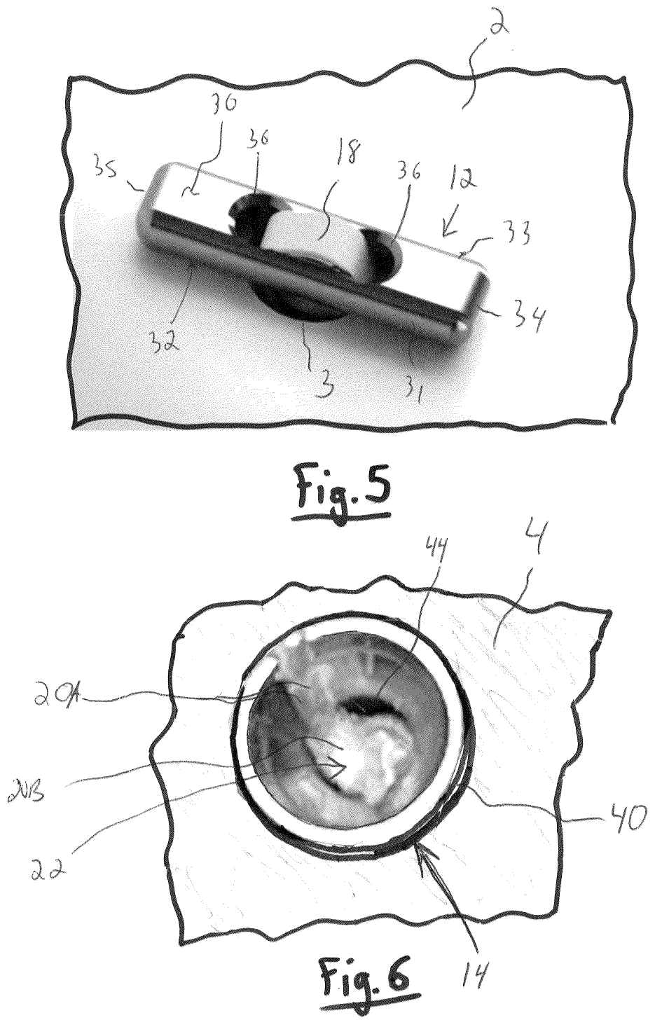

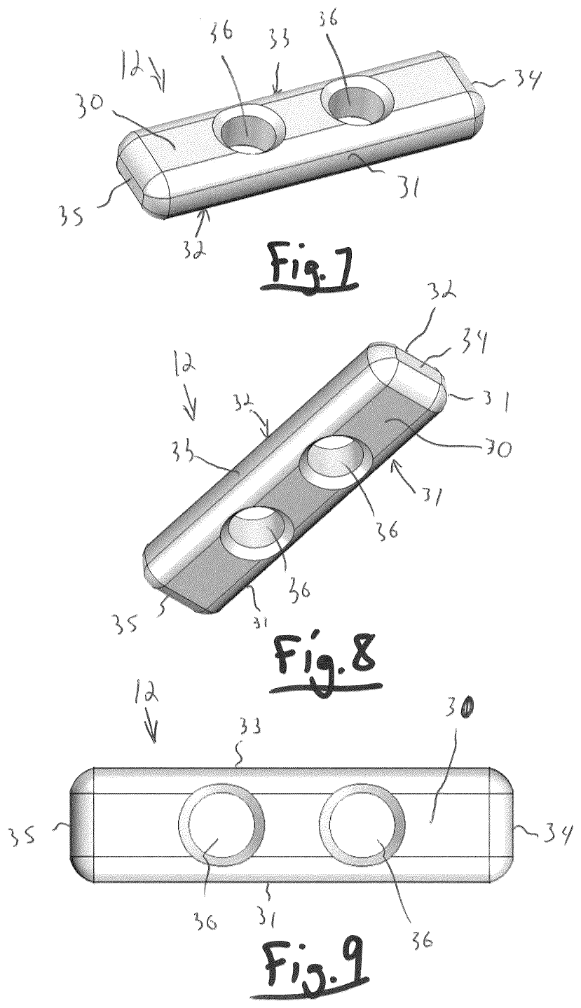

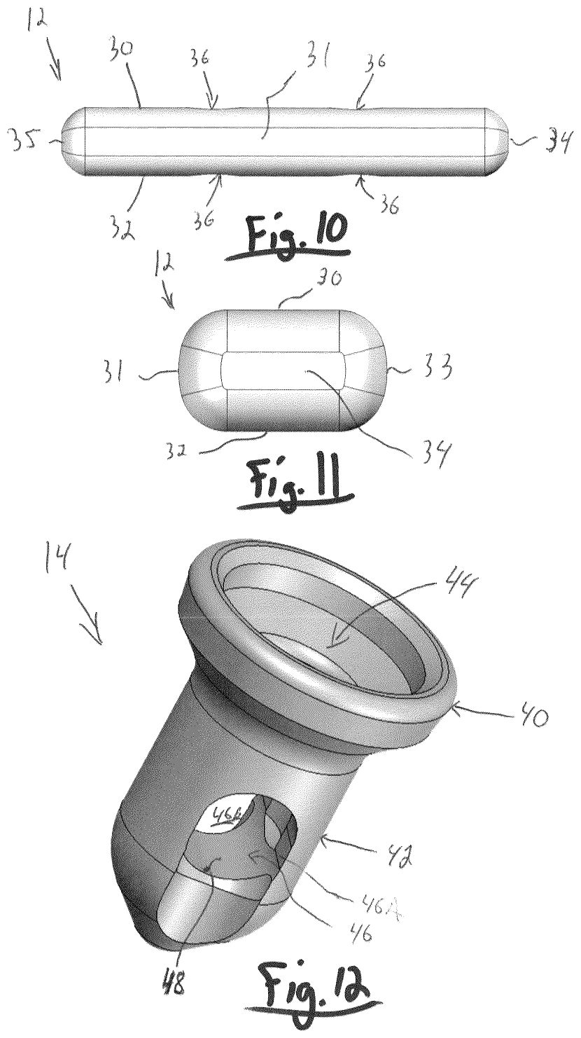

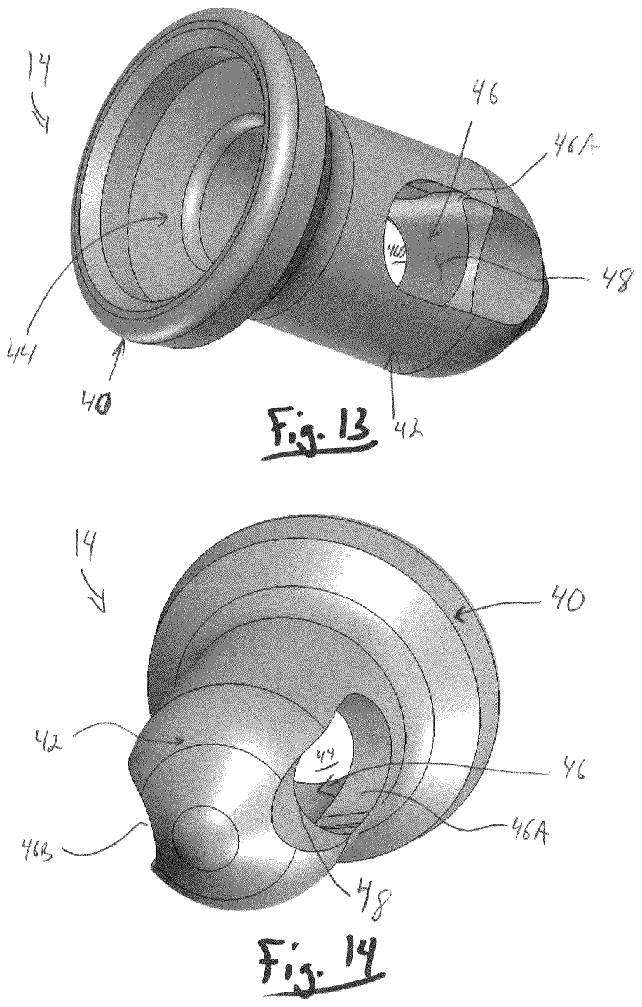

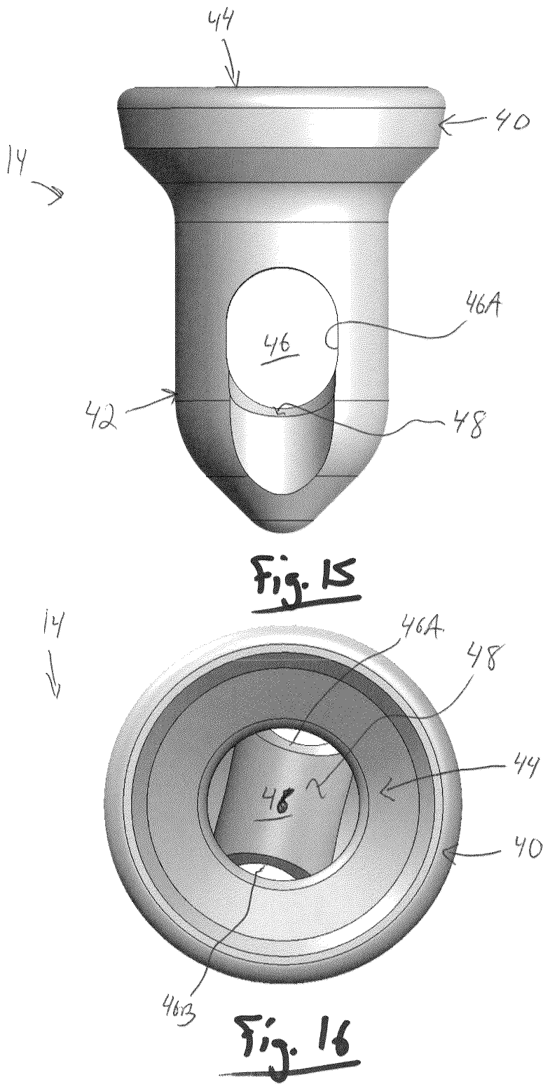

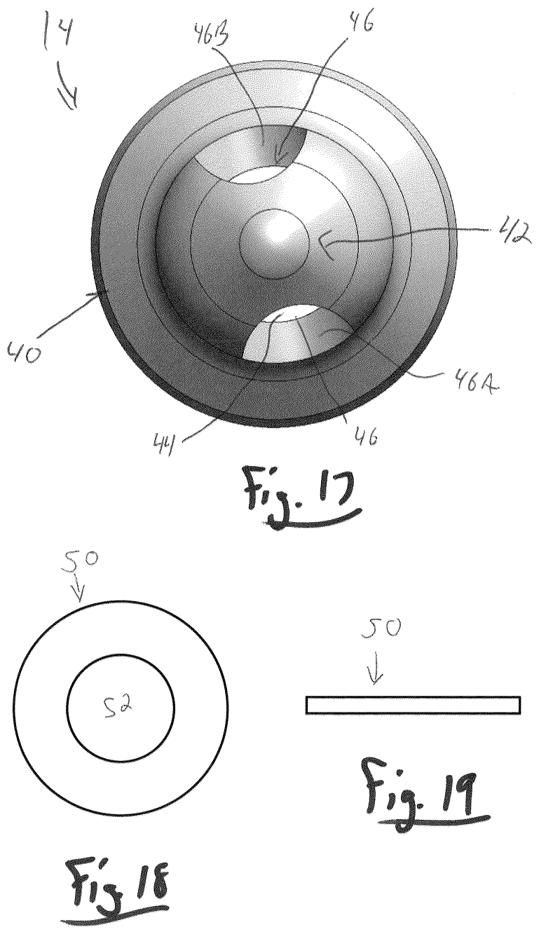

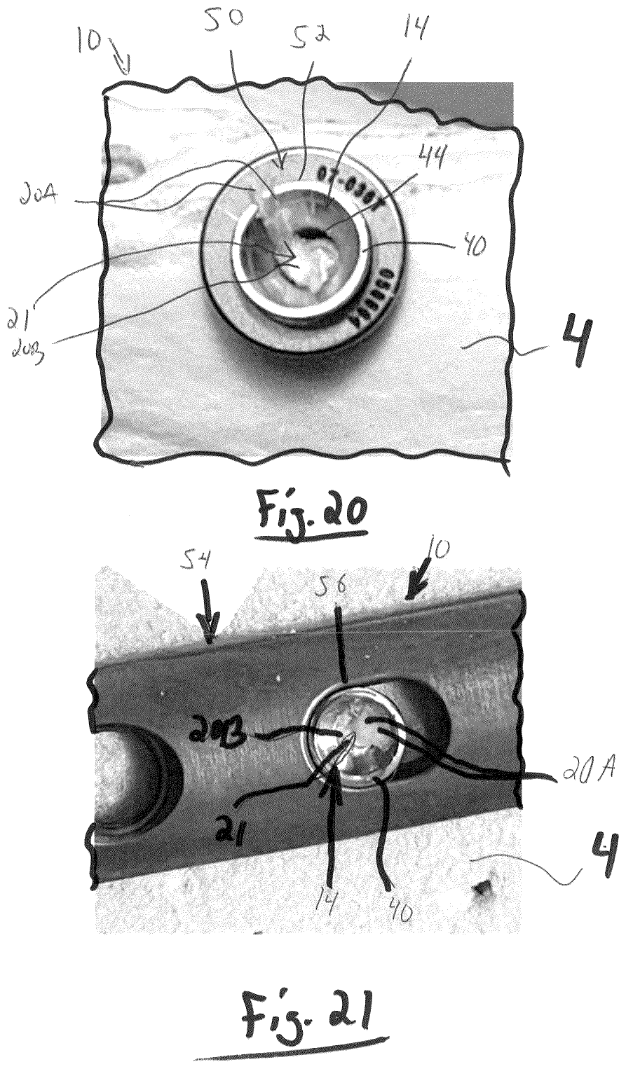

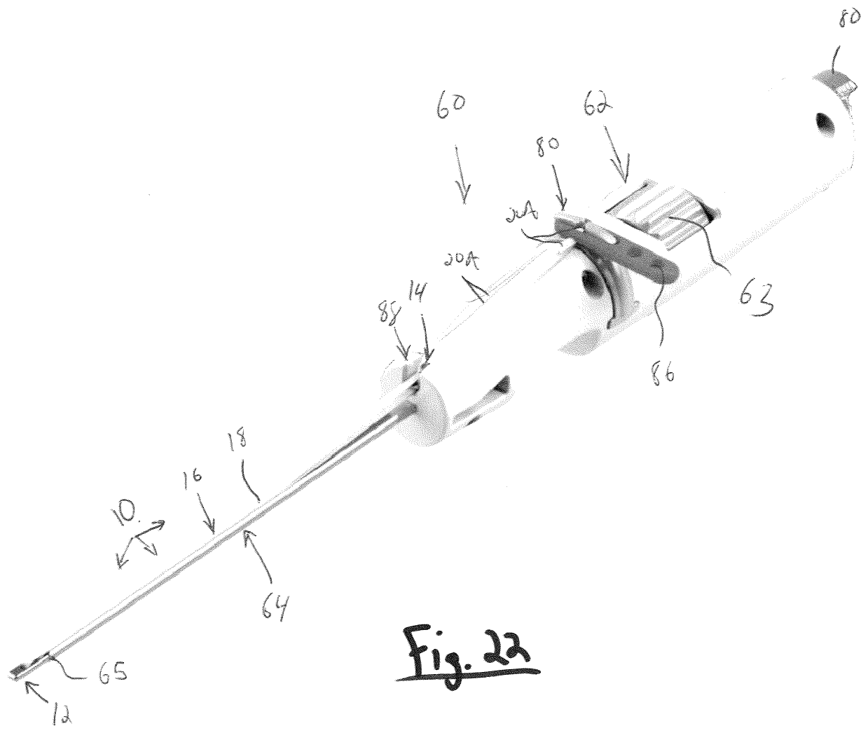

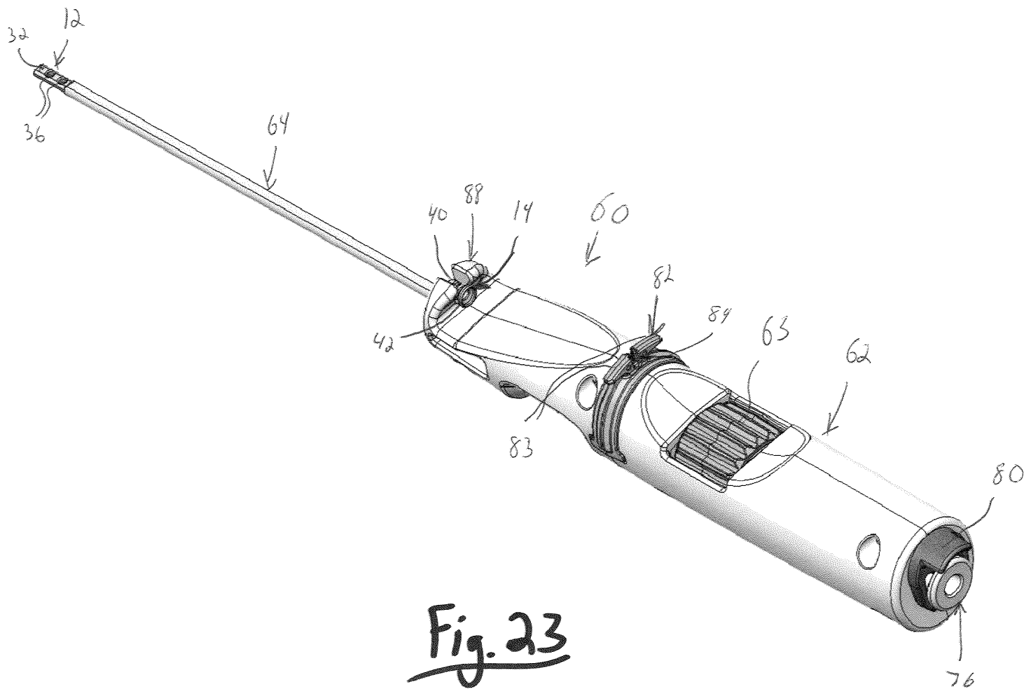

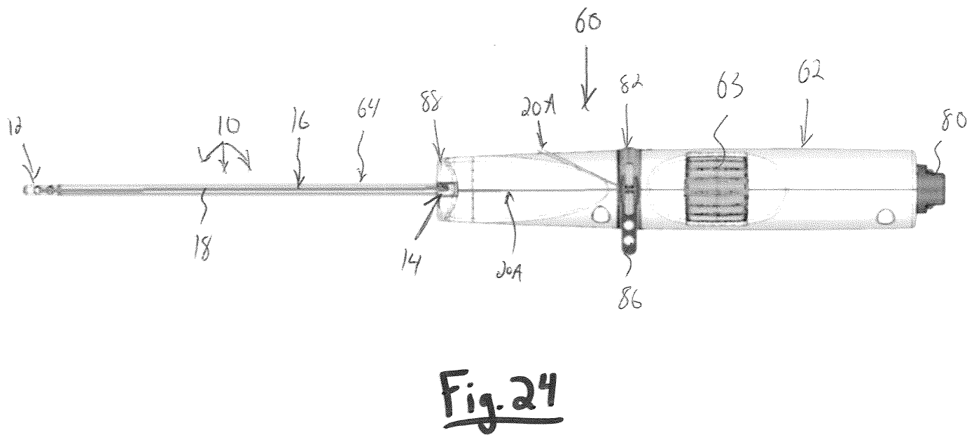

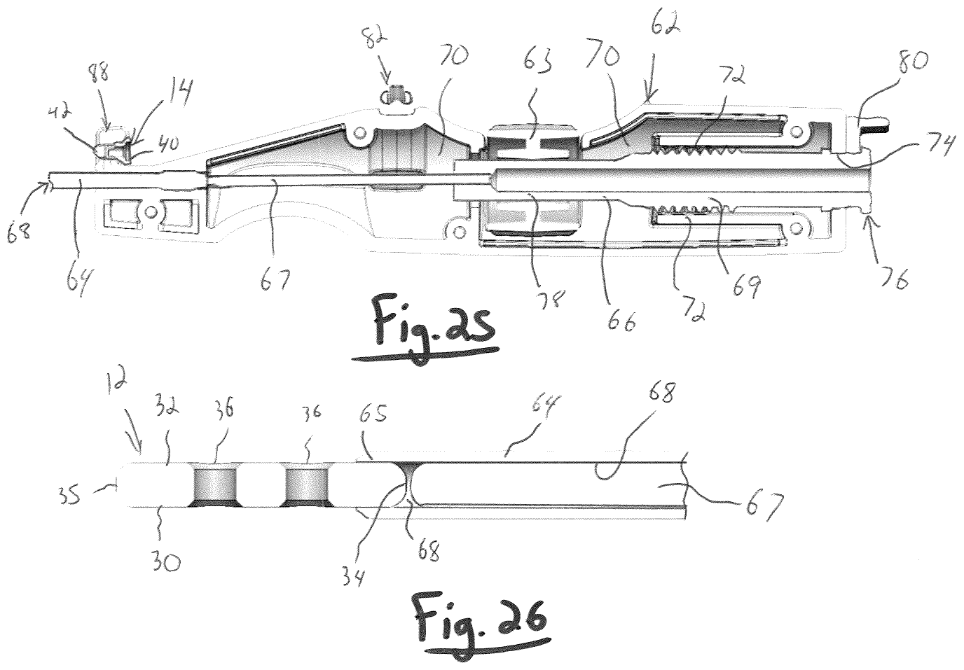

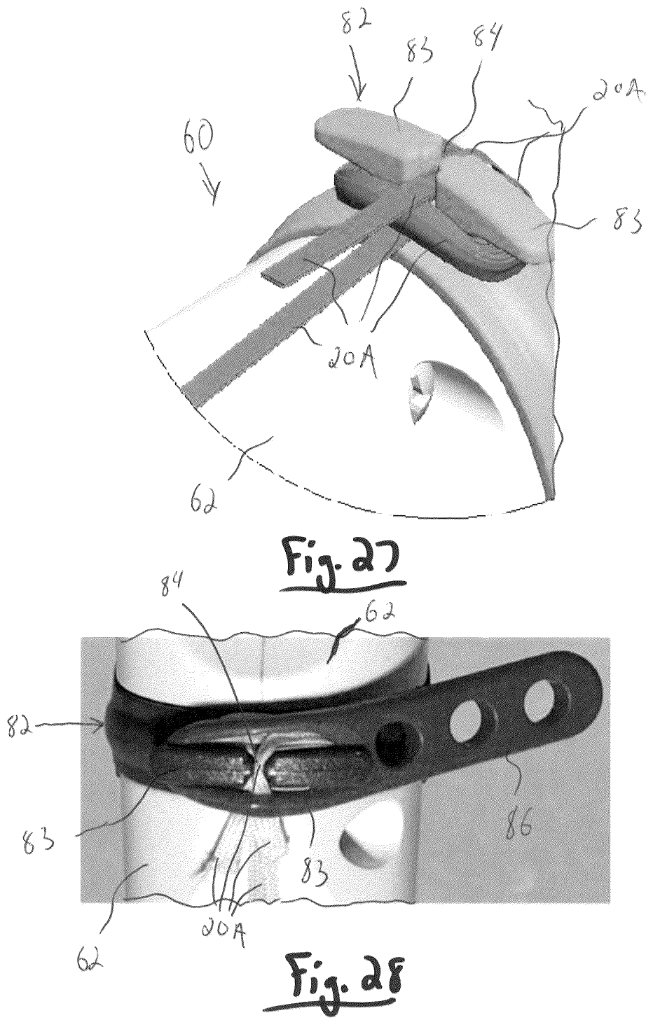

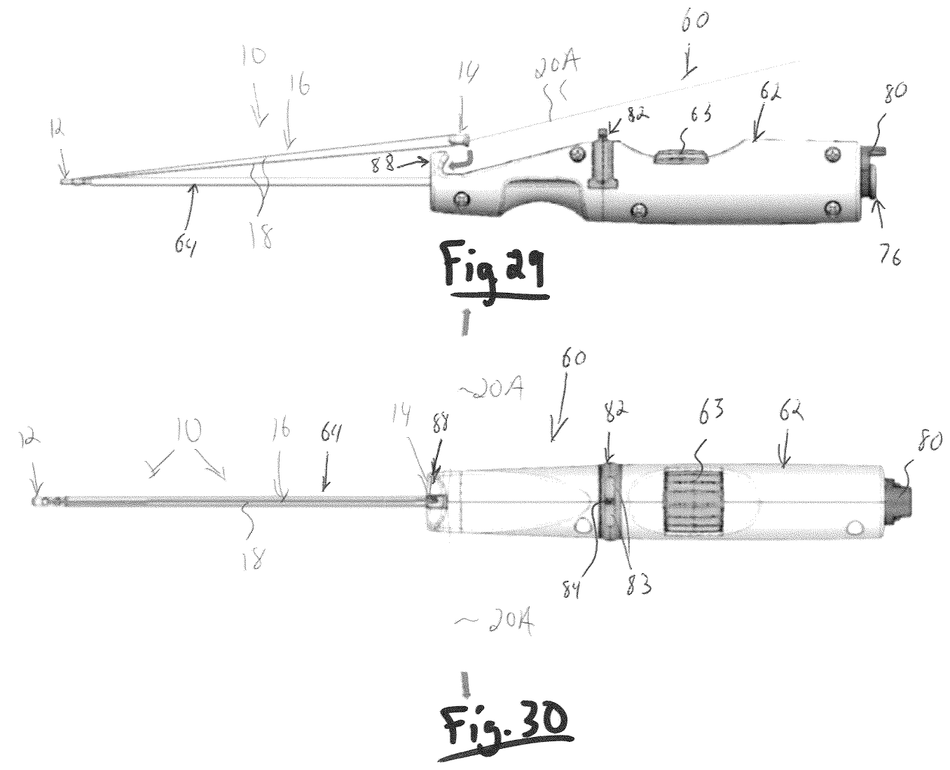

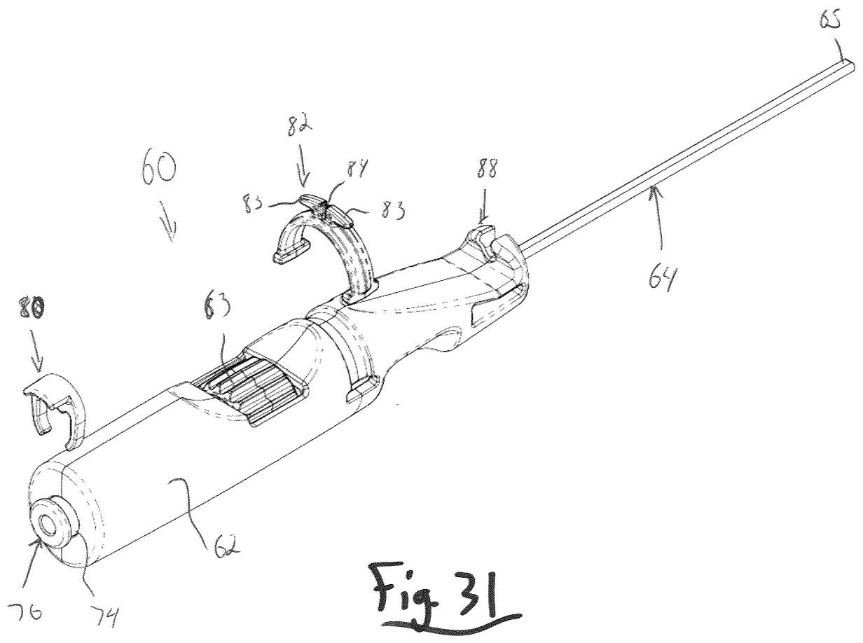

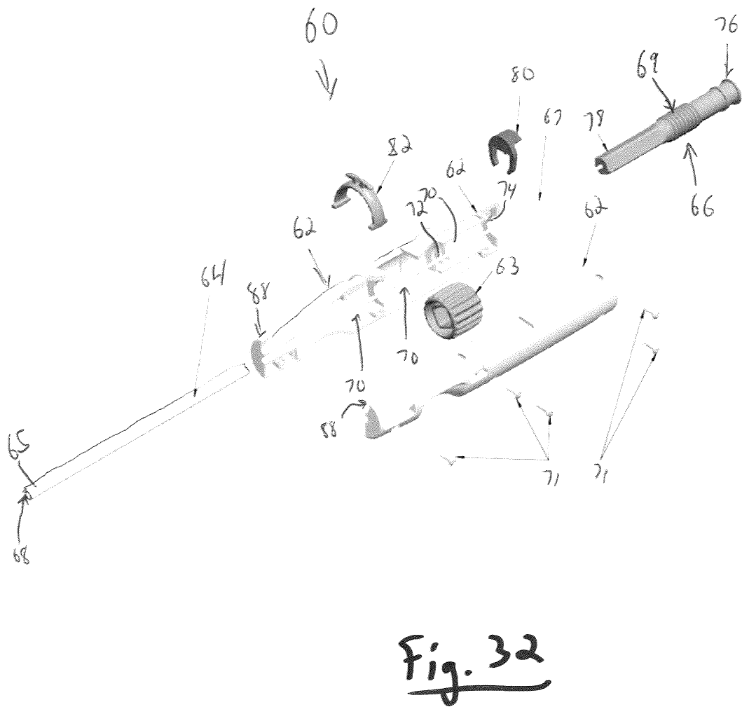

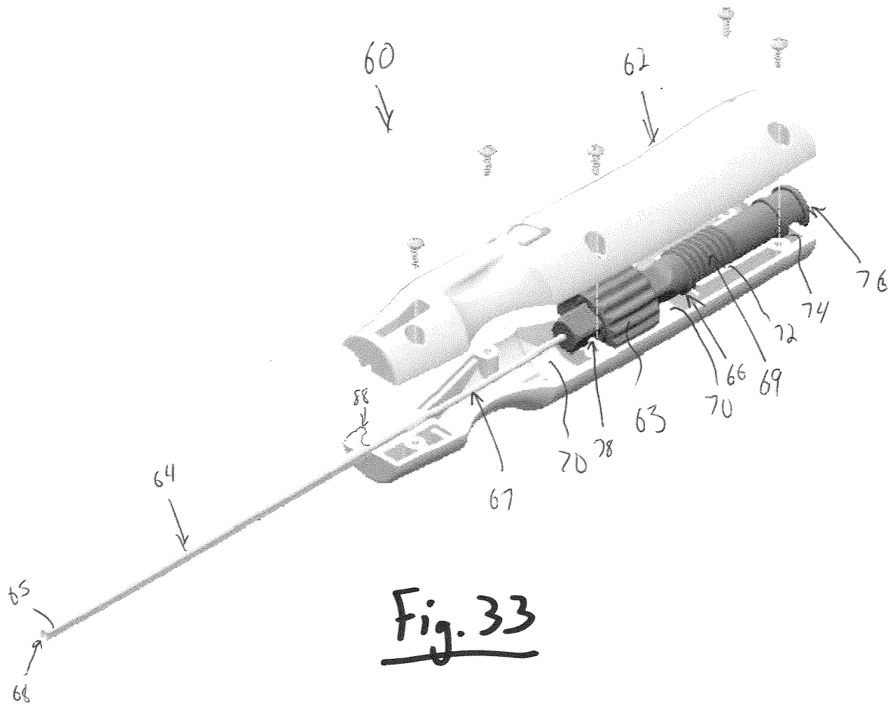

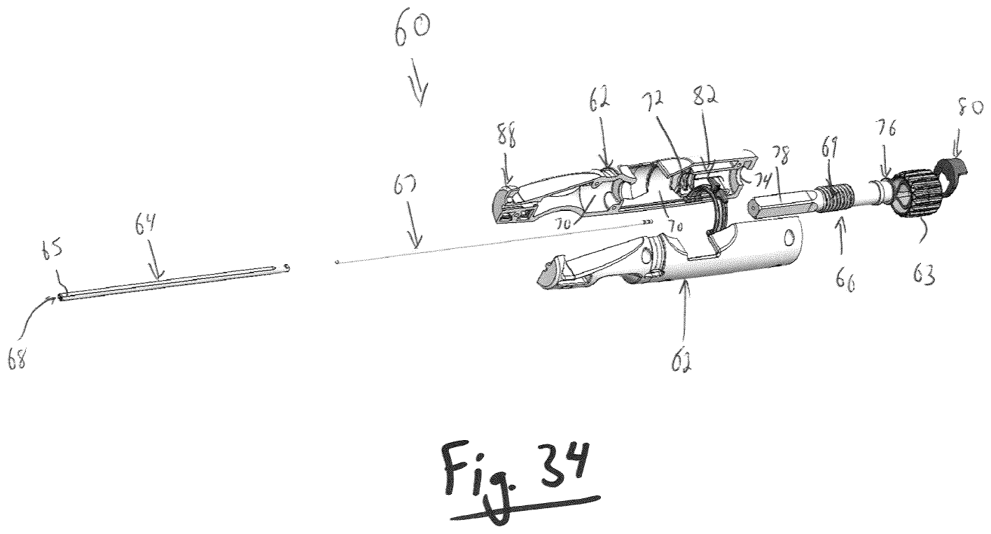

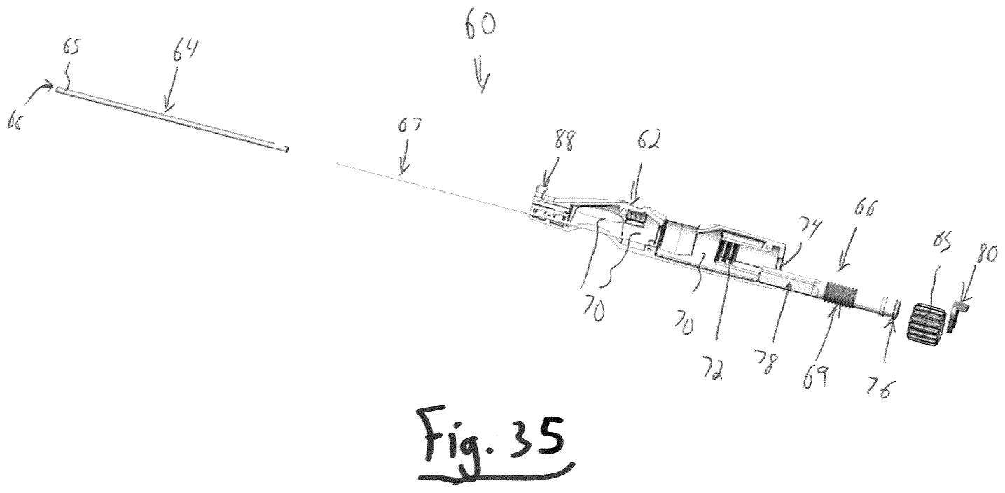

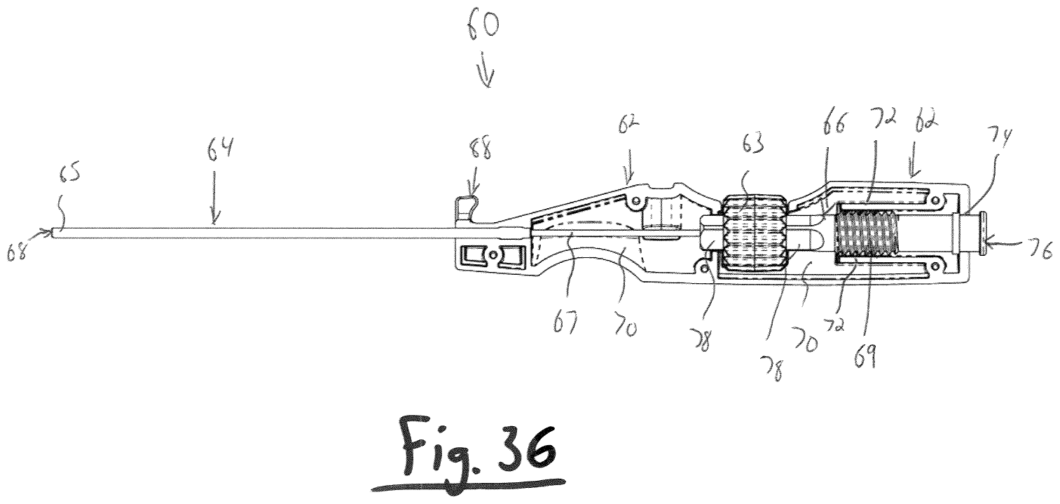

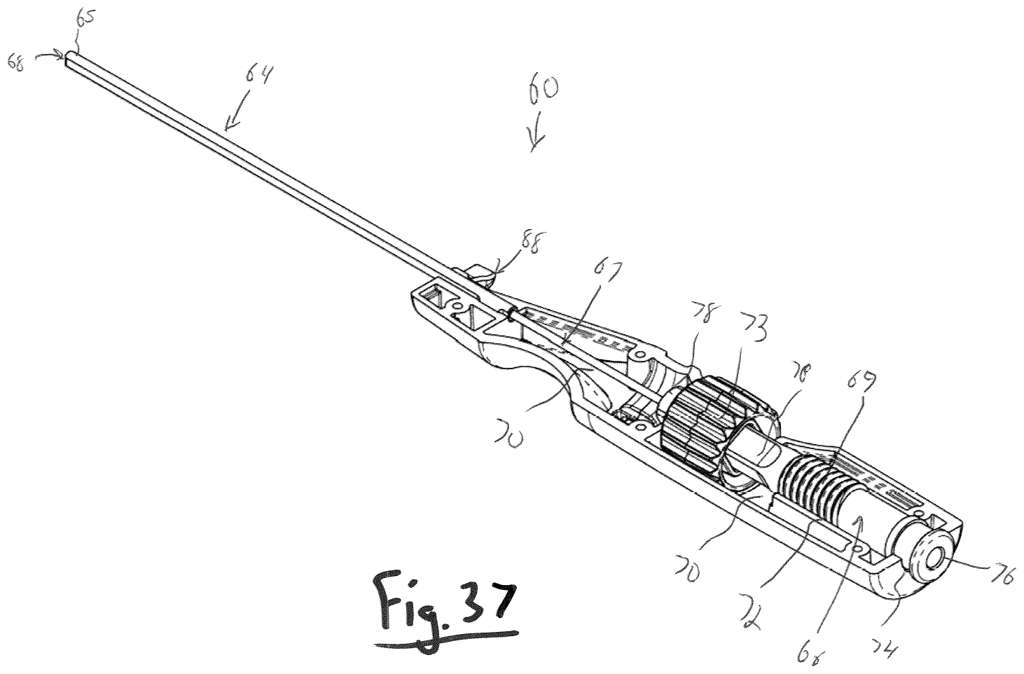

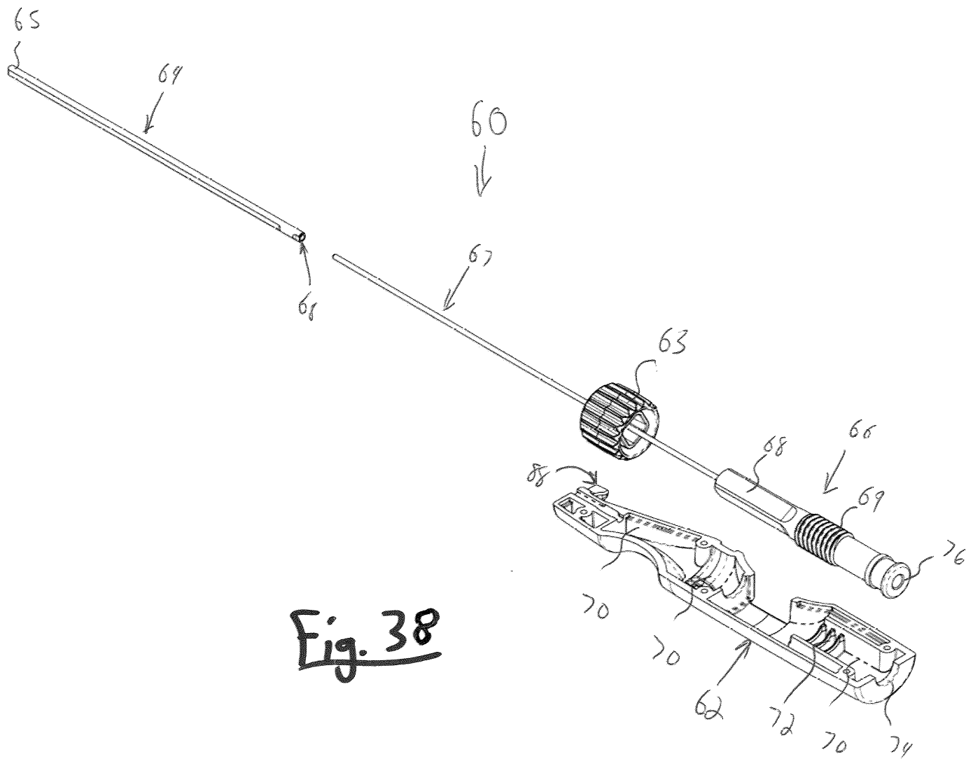

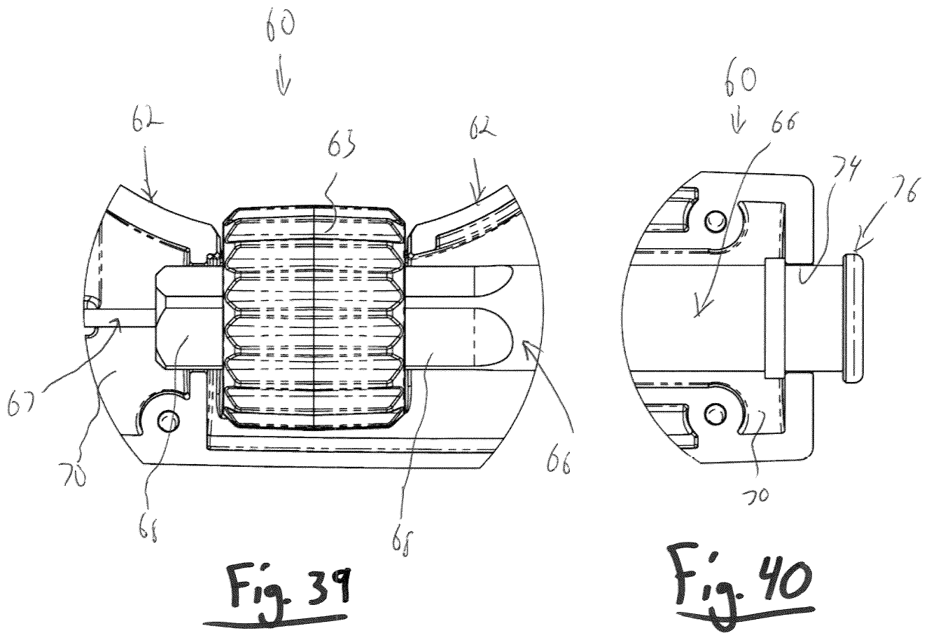

As shown in FIGS. 3, 6 and 20, the adjustment knot 21 may be seated or positioned within a cavity or aperture of the second anchor 14. For example, after the adjustment knot 21 is formed, the intermediate portion 18 may be tensioned (e.g., the first and second anchors 12, 14 pulled/forced away from each other) to force or translate the adjustment knot 21 into a cavity 44 extending into the head portion 40 of the second anchor 14, and within a through hole and against a bearing portion 48 of a stem portion 42 of second anchor 14. The first end portions 20A may extend through the through hole and the cavity 44 and out of (and past) the head portion 40 of the second anchor 14.

With reference to FIG. 4, in one embodiment, the adjustment knot 21 may be formed from the second end loop portion 20B being folded over upon itself such that it forms two loops, which can be arranged or folded next or adjacent to each other (such that the two loops are generally aligned or overlapping). The first end portions 20A of the tether 16 may be passed through the two loops formed from the second end loop portion 20B such that the first end portions 20A extend therethrough to form the adjustment knot 21. The second end loop portion 20B may be tensioned or otherwise manipulated such that the two loops (and thereby the adjustment knot 21 itself) are tightened down onto the first end portions 20A to form the one-way sliding adjustment knot 21.

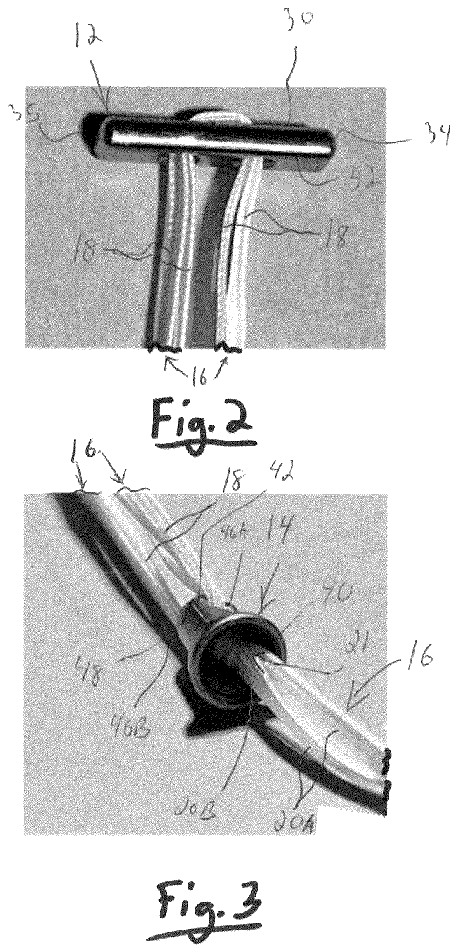

As shown in FIGS. 1, 2, 4 and 5, the intermediate portion 18 of the tether 16 may extend from/between a first aperture or portion 46A of the through hole of the second anchor 14 (e.g., the stem portion 42 thereof) (at least once), through at least one tether aperture 36 of the first anchor 12, and back to/between a second aperture 46B of the second anchor 14 (e.g., the stem portion 42 thereof). The intermediate portion 18 may thereby form a loop portion extending from the second anchor 14 and to and through the first anchor 12. The second anchor 14 may thereby be slidable along the intermediate portion 18. As noted above, the tether 16 may be doubled such that it includes two separate and distinct strands or pieces. In such an embodiment, as shown in FIGS. 2, 4 and 5, both strands of the intermediate portion 18 may extend through the at least one tether aperture 36 of the first anchor 12.

As shown in FIGS. 2, 4, 5 and 7-11, the second anchor 14 may be elongated along a longitudinal direction or dimension (i.e., longitudinally elongated). The second anchor 14 may include or define a maximum longitudinal length or size between a first longitudinal end/side 34 and a second longitudinal end/side 35 that is longer/greater than a maximum lateral width or size between a first lateral end/side 31 and a second longitudinal end/side 33, as shown in FIGS. 5 and 7-11. As also shown in FIGS. 5 and 7-11, the second anchor 14 may include or define a maximum thickness or size between a top outer end/side 30 and a bottom engagement end/side 32 (e.g., a bone and/or tissue engagement end/side) that is less than the maximum longitudinal length (and, potentially, the maximum lateral width).

In some embodiments, the second anchor 14 may extend linearly along the longitudinal direction, as shown in FIGS. 5 and 7-11. For example, in some embodiments, the first lateral end/side 31 and the second longitudinal end/side 33 may extend linearly or straight in the longitudinal direction, and may be substantially parallel to each other. In some embodiments, the bottom engagement end/side 32 and/or the top end/side 30 may extend linearly or straight in the longitudinal direction, and may be substantially parallel to each other. In some the bottom engagement end/side 32 and/or the top end/side 30 may comprise a flat or planar surface. In some embodiments, the edges or junctions of adjoining/adjacent/cooperating the longitudinal, lateral, top and bottom end/side may be rounded or arcuate such that the second anchor 14 is void of sharp or pointed edges.

The second anchor 14 may be configured as a toggle anchor that is configured to physically toggle or rotate/reorient from a longitudinal arrangement where the bottom engagement end/side 32 is angled from a first bone or tissue surface 2 (with the first or second longitudinal end 34, 35 positioned proximate to the first bone or tissue surface 2, and the other of the first or second longitudinal end 34, 35 positioned distal to the first bone or tissue surface 2) to an engagement position with the bottom engagement end/side 32 extending over or along the first bone or tissue surface 2, as shown in FIGS. 5 and 41-46. In the engagement position, the bottom engagement end/side 32 may be in engagement with the first bone or tissue surface 2, and/or extend substantially parallel to the first bone or tissue surface 2, as shown in FIG. 5.

In some embodiments, the least one tether aperture 36 may extend between the top side 30 and the bottom engagement side 32 of the first anchor 12, as shown in FIGS. 2, 4 and 7-11. As also shown in FIGS. 2, 4 and 7-11, in some embodiments, the first anchor 12 may include a pair of tether apertures 36, 36 extending between the top side 30 and the bottom engagement side 32. The pair of tether apertures 36, 36 may be longitudinally spaced from each other, and may be positioned in a middle portion of the first anchor 12 along the longitudinal direction/dimension. As show in FIGS. 2, 4 and 5, in such embodiments, the intermediate portion 18 of the tether 16 may extend from the first aperture or portion 46A of the through hole of the second anchor 14 (e.g., of the stem portion 42 thereof), to and through a first tether aperture 36 of the pair of tether apertures of the first anchor 12 from the bottom engagement side 32 to the top side 30 of the first anchor 12, over the top side 30 of the of the first anchor 12, to and through a second tether aperture 36 of the pair of tether apertures of the first anchor 12 from the top side 30 to the bottom engagement side 32 of the first anchor 12, and to the second aperture 46B of the second anchor 14 (e.g., of the stem portion 42 thereof).

As shown in FIGS. 1, 3, 6 and 12-17, as discussed above, the second anchor 14 may include a head portion 40 and a stem portion 42 extending from the head portion 40. The stem portion 42 may extend from an underside or bottom portion of the head portion 14 to a free end thereof, and may extend along or define an axis. In some embodiments, the free end of the stem portion 42 may be pointed or tapered. The head portion 40 may be enlarged as compared to the stem portion 42. The head portion 40 and/or the shaft portion 42 may define a circular outer/cross-sectional shape.

The second anchor 14 may include a cavity with a plurality of apertures or openings at the exterior of the second anchor 14. As shown in FIGS. 1, 3, 4, 6 and 12-17, the head portion 40 may include a first longitudinal inner or interior cavity, aperture or opening 44 that extends from the end or top of the head portion 40 toward/to the stem portion 42. The first cavity 44 may extend interiorly within the second anchor 14 from the end of the head portion 40 to a second lateral cavity or through hole 46 extending through the stem portion 42. The second cavity or through hole 46 may extend laterally through the stem portion 42 from differing sides of the stem portion 42 such that it forms a first aperture or opening 46A on a first side of the stem portion 42. The first cavity 44 and the second cavity 46 may be in communication such that they cooperatively form an internal cavity, passageway or opening extending through the second anchor 14, such as from the first opening 46A in the stem portion 42 to the top/free end of the head portion 40 and from the second opening 46B in the stem portion 42 to the top/free end of the head portion 40.

As shown in FIGS. 4 and 12-17, a distal portion of the stem 42 at the bottom side of the cavity 46 may form a distal bearing surface or portion 48 within the stem portion 42. The distal bearing surface/portion 48 may form a surface or portion that faces (at least generally) toward the top or head portion 40 of the second anchor 14. As discussed above, the adjustment knot 21 may abut or be retained by/against the bearing surface/portion 48 such that the adjustment knot 21 is retained in the cavity 46 and coupled with the second anchor 14. Specifically, the first end portions 20A of the tether 16 may extend through the first opening 46A and past the bearing surface/portion 48 (such as through the second cavity 46 and the first cavity 44, and potentially past the head portion 44), and the second end portion 20B of the tether 16 may extend through the second opening 46B and at least to the bearing surface/portion 48 within the second cavity 46, as shown in FIGS. 3 and 4. Further, the first and second end portions 20A, 20B may be arranged such that the adjustment knot 21 is positioned within second cavity 46 and against or adjacent to the bearing surface/portion 48, as shown in FIGS. 3, 6, 20 and 21. The tether 16 may thereby wrap or extend over an end portion of the stem portion 42 and be coupled thereto.

In some embodiments, the bearing surface/portion 48 may include chamfered or sloping (linearly and/or arcuately) outer surfaces or recess at the openings 46Am 46B that accommodate the tether 16, as shown in FIGS. 3, 4 and 12-16. The chamfers or recesses may extend from/to the bearing surface/portion 48 at the outer surfaces of the stem portion 42. The chamfers or recesses may extend from/to the bearing surface/portion 48 distally toward the free end or tip of the stem portion 42, as shown in FIGS. 4 and 12-15. The chamfers or recesses may prevent wear on the tether 16 against the stem portion 42, and the chamfers or recesses and the bearing surface/portion 48 may cause the adjustment knot 21 to tighten on the first end portions 28A when the intermediate portion 18 is tensioned (and thereby prevent the intermediate portion 18 from enlarging and the first and second anchors 12, 14 from translating away from each other).

In some embodiments, the implant 10 may further include (or be configured to be used in conjunction with) a washer member 50 configured for use with the second anchor 14. As shown in FIGS. 18-20, the washer member 50 may comprise a disc shaped member with an inner aperture or through hole 52 extending through a thickness thereof. The through hole 52 may be configured to accept or mate with the head portion 40 of the second anchor 14, and extend outwardly or radially therefrom, as shown in FIG. 20. A bottom surface or side of the washer member 50 may be configured to engage the second bone 4 when the second anchor 14 is implanted therein. In some embodiments, the washer member 50 may be ring shaped, and/or substantially thin and flat. However, the washer member 50 may be otherwise shaped or configured.

In some embodiments, the implant 10 may further include (or be configured to be used in conjunction with) a bone plate 54 configured for use with the second anchor 14. As shown in FIG. 21, the bone plate 54 may comprise a rigid plate member with at least one aperture or through hole 56 extending through a thickness thereof. The through hole 56 may be configured to accept or mate with the head portion 40 of the second anchor 14, and extend outwardly or radially therefrom, as shown in FIG. 21. In some embodiments, the through hole 56 may be a central or centered aperture. A bottom surface or side of the bone plate 54 may be configured to engage the second bone 4, and the second anchor 14 may be configured to implanted in the second bone 4 through the through hole 56 with the head portion 40 in engagement with the bone plate 54. It is noted that the bone plate 54, may form any shape or configured to extending over any bone portion. In some embodiments, the bone plate 54 may be substantially thin, and/or comprise a plurality of through holes 54. In some embodiments, the bone plate 54 may be configured to be coupled to the second bone via at least one pin, screw or like fastening member that extends through a through hole of the bone plate 54 and into the second bone 4.

The present disclosure also provides instruments 60 for implanting or deploying the implant 10, such as the exemplary instrument 60 shown in FIGS. 22-46. The instrument 60 is configured to implant/deploy the implant 10 across or through a portion of the first and second bones (and/or tissues) 2, 4, as shown in FIGS. 41-46. In some embodiments, the implant 10 may be mounted or loaded (pre-loaded) on the instrument 60 as shown in FIGS. 22-30. The implant 10 and the instrument 60 may cooperatively form a system, kit, apparatus or device for stabilizing the first and second bones 2, 4.

As shown in FIGS. 22-40, in some embodiments, the instrument 60 comprises a handle or housing portion 62 and a longitudinally elongated insertion tube 64 extending from a front portion of the handle portion 62 to a tip or free end portion 65. The handle portion 62 may be longitudinally elongated, and may be configured to be grasped by a user's hand. The handle portion 62 may form a housing, base or frame that defines at least one internal cavity 70 that houses or contains (at least partially) components of the instrument 60. In some embodiments, at least a rear portion of the handle portion 62 may be generally or substantially cylindrical in shape. The handle portion 62 may be substantially rigid, and may be configured to be manually manipulated/utilized to implant the implant system 10 into/with the first and second bones (as shown in FIGS. 41-46 and described further below).

The insertion tube, portion or member 64 may extend from within the interior cavity 70 of the handle portion 62, out a front portion of the handle portion 62, and to the free end/tip portion 65 thereof. As shown in FIGS. 22-40, the insertion tube 64 includes or defines a longitudinal cavity 68 (e.g., an internal cavity) extending from the free end/tip portion 65 to the handle portion 62 (such as to within the interior cavity 70 of the handle portion 62). In some embodiments, the longitudinal cavity 68 may comprise a through hole extending longitudinally through the insertion tube 64. The internal cavity 68 of the insertion tube 64 may thereby be in communication with the interior cavity 70 of the handle portion 62, and may be open or exposed at the free end/tip portion 65. In some embodiments, the insertion tube 64 (and potentially the cavity 70) may extend linearly longitudinally from the portion of the handle portion 62 to the free end/tip portion 65.

The insertion tube 64 may be substantially longitudinally elongate, as shown in FIGS. 22-40. The insertion tube 64 may be configured to extend through a longitudinal tunnel, hole or cavity in the first and second bones 2, 4, such as cylindrical hole or tunnel formed via drill bit or like tool. The insertion tube 64 may be rigid, and define a cross-sectional size that is substantially smaller than the longitudinal length thereof. In some such embodiments, the insertion tube 64 may define a lateral width and/or thickness that are less that about 1 inch, or less than about ½ inch, or less than about ⅓ inch, or less than ¼ inch. The insertion tube 64 may be fixedly coupled with the handle portion 62. For example, as shown in FIGS. 22-40, the insertion tube 64 longitudinally fixedly coupled to the front portion of the handle portion 62, and potentially rotationally fixedly coupled to the front portion of the handle portion 62.

In a pre-deployment or implantation state of the instrument 60, as shown in FIGS. 22-30, 41, 44 and 45, at least a portion of the first anchor 12 may be received, retained, housed or positioned within the end/tip portion 65 of the cavity 68 of the the insertion tube 64, as shown in FIGS. 22-30, 41 and 43-45. For example, the first anchor 12 may be received, loaded or retained on the instrument 60, in a pre-deployment or implantation state of the instrument 60, such that a longitudinal portion of the first anchor 12 extending from one of the longitudinal end 34, 35 to/toward an adjacent tether aperture 36 is positioned within the end/tip portion 65 of the cavity 68 of the insertion tube 64, and the remainder of the first anchor 12 extends longitudinally therefrom, as shown in FIGS. 22-30, 41 and 43-45. In the pre-deployment or implantation state of the instrument 60, the first anchor 12 may thereby be longitudinally oriented with respect to the longitudinally elongated cavity 68 of the insertion tube 64, and at least a portion of the first anchor 12 along the longitudinal length thereof be removably received within a portion of the longitudinally elongated cavity 68 of the insertion tube 64 at the end/tip portion 65 thereof.

As shown in FIGS. 22-38, the instrument 60 may include an anchor retention portion 88 configured to removably retain the second anchor 14 therein (potentially while the first anchor 12 is removably retained in the insertion tube 64, and after deployment of the first anchor 12 from the insertion tube 64). In some embodiments, the anchor retention portion 88 may be formed via a portion of the handle/housing portion 62. In some embodiments, the anchor retention portion 88 may be positioned at longitudinal front portion of the housing 62 proximate to the insertion tube 64.

In some embodiments, the anchor retention portion 88 may form an opening or cavity (e.g., a longitudinal opening) configured to accept at least the stem portion 42 of the second anchor 14 therein, but prevent the second anchor 14 from longitudinally translating/passing therethrough (e.g., prevent the head portion 40 of the second anchor 14 from longitudinally translating/passing therethrough), as shown in FIGS. 22-30. The anchor retention portion 88 may also comprise a longitudinal slot or passageway extending from a top end or side of the anchor retention portion 88, and from a longitudinal front side of the anchor retention portion 88, to the opening or cavity configured to accept or allow the intermediate portion 18 of the tether 16 to extend between the second anchor member 14 retained in the opening/cavity and the first anchor member 12 (mounted at the tip 65 of the insertion tube 64 and/or deployed therefrom), as shown in FIGS. 22-23.

The anchor retention portion 88 may be configured to only allow translation of the second anchor 14 relative to the anchor retention portion 88, when positioned within the opening/cavity thereof, in a longitudinal direction extending away from the tip 65 of the insertion tube 64. In this way, in the pre-deployment/implantation state of the instrument 60, the first anchor 12 may be seated within and retained by the tip portion 65 of the insertion tube 64, and the second anchor 14 may be seated within and retained by the anchor retention portion 88, via adjustment of the adjustment knot 21 such that the intermediate portion 18 of the tether 16 is of a length that prevents relative longitudinal movement of the first and second anchors 12, 14 away from each other to such an extent that the first anchor 12 can longitudinally translate out from the insertion tube 64 and/or the second anchor 14 can longitudinally translate out from the anchor retention portion 88.

As discussed above, the adjustment knot 21 may be configured to prevent or resist such elongation of the intermediate portion 18, but provide for shortening of the intermediate portion 18 via tension applied to the strands of the first end portions 20A (such as in a direction extending laterally away from each other, as shown in FIG. 30), as shown in FIGS. 22-30. In some embodiments, in the pre-deployment/implantation state of the instrument 60, the first anchor 12 is positioned within the tip portion 65 of the insertion tube 64 and the second anchor 14 is positioned within the the anchor retention portion 88, and the intermediate portion 18 of the tether 16 is in longitudinal tension between the first and second anchors 12, 14 to selectively retain the first anchor 12 within the cavity 68 of the insertion tube 64 and the second anchor 14 within the retention opening of the anchor retention portion 88 until after deployment of the first anchor 12 (as explained further below). It is also noted that tension applied to the first anchor 12 during deployment of the first anchor 12 may act to rotate/reorient the first anchor 12 from the longitudinal arrangement where the bottom engagement end/side 32 is angled from the first bone or tissue surface 2 to an engagement position with the bottom engagement end/side 32 extending over or along the first bone or tissue surface 2, as shown in FIGS. 5 and 41-46.

In some embodiments, the instrument 60 may include a tether retainer portion or member 82, and potentially a tether retainer keeper 86, configured to couple with and retain the first end portions 20A of the tether 16, such as when the second anchor 14 is retained/positioned in the anchor retention portion 88, as shown in FIGS. 22-25 and 27-31.

With reference to FIGS. 27 and 28, in some embodiments, the tether retainer portion 82 may be removably coupled with the housing 62, and/or the tether retainer keeper 86 may be removably coupled with the tether retainer portion 82. In some other embodiments, the tether retainer portion 82 may be fixed to, or formed by part of, the housing 62. In some embodiments, the tether retainer portion 82 may include at least a pair of cleat members or wing projections 83 and a slot 84 extending between the wing portions 83. The wing portions 83 may include a recess or undercut that is configured such that the first end portions 20A of the tether 16 can wrap about a lower portion of the wing portions 83 and a higher top portion of the wing portions 83 extends thereover to prevent the wrapped first end portions 20A from sliding/translating off of the wing portions 83, as shown in FIG. 27. As also shown in FIG. 27, the slot 84 is configured to allow a free end portion of the first end portions 20A to pass therethrough, potentially longitudinally, and be exposed along the handle portion 62 (e.g., not be positioned beneath a higher top portion of one of the wing portions 83).

As shown in FIG. 28, the tether retainer keeper 86 may configured to removably couple with the tether retainer portion 82 over the first end portions 20A of the tether wrapped around the wing portions 83 to prevent un-wrapping or un-raveling of the first end portions 20A from the tether retainer portion 82. For example, the tether retainer keeper 86 may configured to removably couple about the wing portions 83 beneath the higher top portions of the wing portions 83 (and over the first end portions 20A). The tether retainer keeper 86 may also retain or trap the free ends of the first end portions 20A against an outer surface of the instrument 60, such as the outer surface of the housing 62. In some embodiments, the tether retainer keeper 86 may comprise or form an elastic member with an opening that is configured to extend/over about wing portions 83 over the first end portions 20A and beneath the higher top portions of the wing portions 83 in a resiliently deformed expanded state. The tether retainer keeper 86 may thereby be elastically deformed into an enlarged state (e.g., stretched) to remove the tether retainer keeper 86 from over/about the wing portions 83 and the first end portions 20A.

As shown in FIGS. 22-64, the instrument 60 may further comprise a deployment portion that is threadably engaged with a portion of the instrument 60 and configured to decouple or deploy the first anchor 12 from within the cavity 68 of the insertion tube 64 from the pre-deployment/implantation state of the instrument 60 (via the threaded connection). With reference to FIGS. 32-40, in some embodiments, the deployment portion of the instrument 60 may comprise a longitudinally elongated deployment rod portion or member 67 translatably received within the longitudinally elongated cavity 68 of the insertion tube 64, a base portion or member 66 that is coupled with the deployment rod 67 and extending within the cavity 70 of the handle portion 62, and a manually engageable adjustment portion or member 63 coupled with the base member 66 and extending exterior to the housing portion 62. As shown in FIGS. 32-46, the deployment portion is configured such that movement of the adjustment member 63 in a first direction adjusts the threaded engagement of the deployment portion with respect to the instrument 60 to drive the deployment rod portion 67 longitudinally within the cavity 68 of the insertion tube 64 toward the tip/end portion 65 thereof to, ultimately, force the first anchor 12 out from the cavity 68 (i.e., decouple, deploy or implant the first anchor 12 from the pre-deployment/implantation state of the instrument 60).

As shown in FIGS. 32-39, the deployment rod portion 67 may be longitudinally slidably or translatably received within the cavity 68 of the insertion tube 64. The deployment rod 67 extends within the cavity 68 of the insertion tube 64 from the handle portion 62 (e.g., from within the cavity 70 of the handle portion 62) to the end/tip portion 65. The deployment rod 67 may be configured to translate longitudinally toward, and longitudinally away from, the end/tip portion 65 of the insertion tube 64 within the cavity 68 to the handle portion 62 (depending upon the direction of the force) via the threaded connection of the deployment portion and selective movement (e.g., selective manual movement) of the adjustment member 63 relative to the housing 62, as shown in FIGS. 32-46. The deployment rod 67 may be rigid or stiff and otherwise configured such that longitudinal force applied to a base portion of the deployment rod 67 within/at the handle portion 62 is effective in longitudinally translating the deployment rod 67 within the cavity 70 toward or away from the end/tip portion 65.

A longitudinal rear or base portion of the deployment rod portion 67 may be coupled or engaged with the base member portion 66, such as with a longitudinal front portion of the base member portion 66 within the internal cavity 70 of housing 62, as shown in FIGS. 25, 32, 33 and 36-39. The deployment rod portion 67 and the base member portion 66 may be coupled such that longitudinal movement of the base member portion 66 toward the insertion tube 64 effectuates longitudinal movement of the deployment rod portion 67 within the cavity 68 toward the tip/end portion 65 (and potentially toward the first anchor 12). The deployment rod portion 67 and the base member portion 66 can be coupled such that rotation of the base member portion 66 about a longitudinal axis thereof rotates or does not rotate the deployment rod portion 67 about the axis. In some embodiments, the deployment rod portion 67 and the base member portion 66 may be longitudinally fixedly coupled, and potentially also rotationally fixedly coupled.

In some embodiments, the deployment portion is threadably engaged with the portion of the instrument 60 via the base portion 66 being threadably engaged with a threaded drive portion 72 of the housing portion 62, such as an inner threaded drive portion 72 of the housing portion 62, as shown in FIGS. 25 and 32-39. In some such embodiments, a drive portion 69 of the base portion 66 may include external threads, and the drive portion 72 of the housing portion 62 may include interior threads threadably mated with the external threads of the drive portion 69 of the base portion 66. The base portion 66 is rotatably and longitudinally movably received within the cavity 70 of the housing/handle portion 62, and the drive portions 69, 72 are configured, such that rotation of the base portion 66 about an axis thereof (which may be longitudinally oriented) threadably drives/translates the base portion 66 longitudinally with respect to the housing 62 and the insertion tube 64, which thereby drives/translates the deployment rod portion 67 longitudinally with respect to the insertion tube 64.

As shown in FIGS. 25 and 32-39, adjustment member 63 may be movably coupled to the housing portion 62 indirectly via other portions or components of the deployment portion. For example, the adjustment member 63 may be coupled with the base portion 66, and the base portion 66 may be retained or held within the cavity 70 of the housing portion 62 and threadably coupled with the threaded driving portion 72 of the housing portion 62 (which may be an interior portion of the housing portion 62), as described above. In one exemplary embodiment, as shown in FIGS. 25 and 29-39, the adjustment member 63 is configured as a manual rotation member (e.g., a thumb wheel) that extends from within the cavity 70 of the housing 62 to exterior to an outer surface of the housing 62.

The adjustment member 63 may be rotationally fixedly coupled with the base portion 66, and longitudinally movably coupled with the base portion 66. For example, as shown in FIGS. 25 and 32-39, the adjustment member 63 may be rotationally fixedly coupled and longitudinally movably coupled with a torque portion 78 of the base portion 66. In some embodiments, the threaded drive portion 69 of the base portion 66 may be longitudinally positioned between the torque portion 78 and a back end portion 76 of the base portion 66, as shown in FIGS. 25 and 32-39. The torque portion 78 may be longitudinally extended such that it remains drivingly engaged/rotationally fixed with the adjustment member 63 as it longitudinally translates with respect to the adjustment member 63, as explained further below. In some embodiments, the torque portion 78 may be a front end portion of the base portion 66.

In some embodiments, the adjustment member 63 may include a drive aperture or through hole of a non-circular cross sectional shape (with respect to a rotation axis), and the torque portion 78 of the base portion 66 may include a non-circular cross sectional shape (with respect to a rotation axis) that mates within the drive aperture of the adjustment member 63, as shown in FIGS. 25 and 32-39. The torque portion 78 and the adjustment member 63 may thereby be rotationally locked or fixed together about the rotation axis (which may extend longitudinally, and may be aligned with a longitudinal axis of the instrument 60 and/or insertion tube 64). Further, the torque portion 78 and the adjustment member 63 may thereby be longitudinally slidably coupled such that the torque portion 78 and the adjustment member 63 is able to longitudinally slide or translate through the drive aperture adjustment member 63. The deployment portion may be configured such that rotation of the adjustment member 63 in a first rotational direction about a rotation axis (which may be a longitudinal rotation axis) rotates the base portion 66 with respect to the housing portion 62 (specifically the driving portion 72 thereof), and thereby threadably drives the base portion 66 and the deployment rod portion 67 longitudinally with respect to the adjustment member 63, the housing 62 and the insertion tube 64 (via the driver portions 69, 72) such that the deployment rod portion 67 is longitudinally translated within the cavity 68 of the insertion tube 64 toward the tip/end portion 65 of the insertion tube 64 to force the first anchor 12 out from the insertion tube 64, as shown in FIGS. 43 and 44.

As shown in FIGS. 22-25, 29-38 and 40, the back end portion 76 of the base portion 66 extends through a back end opening or aperture 74 of the housing/handle portion 62 such that a portion of the back end portion 76 is positioned past the back end of the housing portion 62. A rear portion of the back end portion 76 positioned past the back end of the housing portion 62 may be an enlarged portion that defines a larger cross-sectional size than the adjacent portion of the back end portion 76 (e.g., the portion of the back end portion 76 that extends through the back end opening 74), as shown in FIG. 40. In some such embodiments, the enlarged portion of the back end portion 76 may define a larger cross-sectional size than the back end opening 74 to prevent the back end portion 76 from longitudinally translating further into the cavity 70 of the housing 62 to limit/stop the longitudinal travel of the base portion 66, and thereby limit/stop the longitudinal travel of the deployment rod portion 67 in the insertion tube 64 toward the tip/end portion 65 (e.g., to prevent the deployment rod portion 67 from extending past the tip/end portion 65), as shown in FIG. 40.

In some embodiments, the instrument 60 may include a safety clip 80 removably coupled (e.g., resiliently) with the portion of the back end portion 76 of the base portion 66 that extends past the rear end of the housing 62, shown in FIGS. 22-25, 29-38 and 40. The safety clip 80 may be positioned longitudinally between the rear end of the housing 62 and the enlarged portion of the back end portion 76 to prevent longitudinal translation of the base portion 66 toward the insertion tube 64. The safety clip 80, when installed, may thereby prevent premature or accidental deployment of the first anchor 12 from the insertion tube 64 via longitudinal translation deployment rod portion 67 (via longitudinal translation of the base portion 66, such as via movement of the adjustment member 63).

FIGS. 41-46 illustrate exemplary methods of utilizing the implant 10 and the instrument 60 to implant or deploy the implant 10 across first and second bones/tissues 2, 4 to stabilize the first and second bones/tissues 2, 4. As noted above, and shown in FIGS. 41 and 45, in the pre-deployment/implantation state of the instrument 60, the first anchor 12 is positioned within the tip portion 65 of the insertion tube 64 and the second anchor 14 is positioned within the anchor retention portion 88, and the intermediate portion 18 of the tether 16 extending between the first and second anchors 12, 14 (potentially in tension) (and potentially the end portions 20A coupled with the tether retainer portion 82) to selectively retain the first anchor 12 within the cavity 68 of the insertion tube 64 and the second anchor 14 within the retention opening of the anchor retention portion 88 for implantation/deployment of the implant 10. As shown in FIG. 41, in some embodiments, in the pre-deployment/implantation state of the instrument 60, the instrument 10 may further include the washer member 50 mounted on the insertion tube 63 over the intermediate portion 18 of the tether 16 (e.g., the insertion tube 63 over the intermediate portion 18 extending through the through hole 52).

As shown in FIGS. 41-44, in some embodiments, a through hole or tunnel may be formed through a portion of the first and second bones/tissues 2, 4 in preparation for the implantation of the implant 10 (mounted on the instrument 60 with the instrument 60 in the pre-deployment/implantation state of the instrument 60). As shown in FIG. 41, the instrument 10 may be manually manipulated and utilized to pass/translate the first anchor 12 (and the insertion tube 64) through the tunnel from an outer side of the second bone/tissue 4 and through a portion of the first and second bones/tissues 2, 4 such that the first anchor 12 is positioned past an outer side of the first bone/tissue 2 and the second anchor 14 is positioned at/adjacent to, but spaced from, the outer side of the second bone/tissue 4. If the washer member 50 is utilized, the washer member 50 may be positioned between the outer side of the second bone/tissue 4 and the second anchor 14, as shown in FIG. 41.

In some embodiments, the first and second bones 2, 4 are a distal tibia and a distal fibula, respectively, that form a distal tibiofibular syndesmosis joint. In such embodiments, the outer side of the first bone 2 may be a medial side of the distal tibia (e.g., a medial tibia cortex), and the outer side of the second bone 4 may be a lateral side of the distal fibula. It is noted that in such a configuration, a medial incision is not required to implant the implant 10.

As shown in FIGS. 42-44, after the first anchor 12 is passed through the tunnel in the first and second bones 2, 4 and past the outer side of the second bone/tissue 4, the safety clip 80 may be removed from the back end portion 76 of the base portion 66, and the adjustment member 63 may be manually moved/adjusted in the first direction (e.g., rotated clockwise or counterclockwise) to longitudinally advance/translate the deployment rod portion 67 through the cavity 68 of the insertion tube 64 toward the tip/end portion 65 until the longitudinal end/tip of the deployment rod portion 67 engages and longitudinally advances/translates the first anchor 12 through the tip/end portion 65 and out from the cavity 68. As discussed above, tension in the intermediate portion 18 of the tether 16 may toggle or rotate the first anchor 12 from a longitudinal arrangement with the engagement surface 32 thereof angled to the outer side of the second bone/tissue 4 to an engagement/deployed arrangement with the engagement surface 32 extending over and potentially in abutment/engagement with outer side of the second bone/tissue 4, as shown in FIGS. 42-44.

After the first anchor 12 is deployed/implanted onto/at the outer side of the second bone/tissue 4, the tether retainer keeper 86 may be removed from the tether retainer portion 82 and the first end portions 20A of the tether 16 may be released from the tether retainer keeper 86, and the second anchor 14 may be removed from the anchor retention portion 88. It is noted that after the first anchor 12 is deployed/implanted onto/at the outer side of the second bone/tissue 4, tension in the intermediate portion 18 of the tether 16 may be released, which may allow the removal of the second anchor 14 from the anchor retention portion 88. It is also noted that the release of the tension in the intermediate portion 18 when the first anchor 12 is deployed/implanted onto/at the outer side of the second bone/tissue 4 may be transferred to the adjustment member 63 and tactilely felt by a user.

As shown in FIG. 6, after the second anchor 14 and the end portions 20A are released/decoupled from the instrument 60, the strands of the end portions 20A may be tensioned/pulled (e.g., laterally away from each other) to slide the adjustment knot 21 along the tether 16 and shorten the intermediate portion 16 to translate the second anchor 14 (and potentially the washer member 50) against/into abutment with the outer side of the second bone/tissue 4, and potentially pull together the first and second bones 2, 4 and/or tension the intermediate portion 18.

With the implant 10 reduced and the first anchor 12 seated against the outer side of the first bone/tissue 2 and the second anchor 14 (and/or washer member 50) seated against the outer side of the second bone/tissue 4, the first end portions 20A of the tether 16 may be securely knotted or tied into a securement arrangement/knot 22 to fix the tether 16 at the second anchor 14 and prevent enlargement of the intermediate portion (i.e., the end portions 20A from passing through the adjustment knot 21 and through the second anchor 14), as shown in FIG. 6. For example, the first end portions 20A of the tether 16 may be tied into one or more surgeon knot, square knot or half hitches. Finally, the first end portions 20A of the tether 16 extending past the securement arrangement/knot 22 may be trimmed, as shown in FIG. 6.

As shown in FIGS. 45 and 46, the instrument 10 may be utilized to implant or deploy the implant 10 across first and second bones 2, 4 where the second bone includes a bone plate 54 coupled thereto to stabilize the first and second bones/tissues 2, 4. The method of implanting the implant 10 with the bone plate 54 coupled to the second bone 4 is substantially similar to the method described above with respect to FIGS. 41-44, and therefor common aspects are not repeated here for brevity purposes. As shown in FIGS. 45 and 46, the instrument 60 and implant 10 may be utilized with the bone plate 54 by substituting the aperture 54 of the bone plate 54 at the entrance to the tunnel through the first and second bones 2, 4, and the outer surface of the bone plate 54 as the outer surface of the second bone 4.

As may be recognized by those of ordinary skill in the art based on the teachings herein, numerous changes and modifications may be made to the above-described and other embodiments of the present disclosure without departing from the scope of the disclosure. In addition, the implants and systems may include more or fewer components or features than the embodiments as described and illustrated herein. Accordingly, this detailed description of the currently-preferred embodiments is to be taken as illustrative, as opposed to limiting of the disclosure.

Similarly, positions or directions may be used herein with reference to anatomical structures or surfaces. Further, the implants, systems, devices, instrumentation and methods, and the aspects, components, features and the like thereof, may be disclosed herein are described with respect to one side of the body for brevity purposes. However, as the human body is relatively symmetrical or mirrored about a line of symmetry (midline), it is hereby expressly contemplated that the implants, systems, devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, systems, instrumentation and methods, and the aspects, components, features and the like thereof, described herein with respect to a right syndesmosis joint (right ankle/leg) may be mirrored or otherwise reconfigured, if necessary or desirable, so that they likewise function with a left syndesmosis joint (left ankle/leg). Further, the implants, systems, devices, instrumentation and methods, and the aspects, components, features and the like thereof, disclosed herein are described with respect to a distal tibiofibular syndesmosis, but it should be understood that the implants, systems, devices, instrumentation and methods may be used with other bones of the body having similar structures.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. An implant system for stabilizing first and second bones, comprising:
a stabilization implant, comprising:
a first bone anchor;
a second bone anchor; and
a flexible tether extending between and coupling the first and second anchors; and
an instrument configured to deploy the implant across a portion of the first and second bones, comprising:
a handle portion with a manually engageable adjustment member movably coupled thereto;
a longitudinally elongated insertion tube extending from a front portion of the handle portion to a tip and comprising a longitudinally elongated cavity; and
a deployment portion coupled with the adjustment member and threadably engaged with a portion of the instrument, the deployment portion comprising a base portion extending within the handle portion and a longitudinally elongated deployment rod portion slidably received within the cavity of the insertion tube,
wherein at least a portion of the first bone anchor is removably received within the cavity of the insertion tube at the tip thereof, and
wherein movement of the adjustment member in a first direction adjusts the threaded engagement of the deployment portion with respect to the instrument to threadably drive the deployment rod portion longitudinally within the cavity of the insertion tube toward the tip thereof to force the first bone anchor out from the cavity.

2. The system of claim 1, wherein the deployment portion is threadably engaged with the portion of the instrument via the base portion being threadably engaged with an inner portion of the handle portion.

3. The system of claim 2, wherein the base portion comprises external threads, and the inner portion of the handle portion comprises interior threads threadably mated with the external threads.

4. The system of claim 2, wherein the base portion is rotatably and longitudinally slidably received with the handle portion, and wherein the adjustment member is rotationally fixedly coupled with the base portion.

5. The system of claim 4, wherein the first direction is a rotational direction extending about a longitudinal axis, and wherein rotation of the adjustment member in the first direction rotates the base portion within the handle portion and threadably drives the base portion and the rod portion longitudinally toward the tip.

6. The system of claim 5, wherein the adjustment member is configured as a thumb wheel that extends within the handle portion and exterior to an outer surface of the handle portion.

7. The system of claim 5, wherein the base portion is longitudinally slidably coupled with the adjustment member.

8. The system of claim 1, wherein the base portion of the deployment portion extends through a back end of the handle portion such that a back end portion of the base portion is positioned past the back end of the handle portion, and wherein the instrument further comprises a safety clip removably coupled with the back end portion of the base member that engages with the back end of the handle portion to prevent longitudinal movement of the deployment rod portion within the cavity of the insertion tube toward the tip thereof.

9. The system of claim 1, wherein the base portion of the deployment portion extends through an opening in a back end of the handle portion such that a back end portion of the base portion is positioned past the back end of the handle portion, and wherein at least a first portion of the back end portion of the base portion defines a dimension larger than the opening such that longitudinal movement of the deployment portion toward the tip is prevented when the first portion engages the back end of the handle portion.

10. The system of claim 1, wherein the second bone anchor member is removably retained within a retention opening of an anchor retention portion of the handle portion.

11. The system of claim 10, wherein the tether is in longitudinal tension between the first and second bone anchors to selectively retain the first bone anchor within the cavity of the insertion tube and the second bone anchor within the retention opening of the anchor retention portion until after deployment of the implant.

12. The system of claim 1, wherein the first bone anchor comprises a first pair of longingly spaced tether apertures each extending through a thickness thereof between an engagement side and an outer side thereof, and wherein an intermediate portion of the tether extends through a first tether aperture of the first pair of tether apertures from the engagement side to the outer side, and then through a second tether aperture of the first pair of tether apertures from the outer side to the engagement side.

13. The system of claim 12, wherein the second bone anchor comprises a second pair of tether apertures, and wherein a first end portion of the tether extends through a third tether aperture of the second pair of tether apertures, a second end portion of the tether extends through a fourth tether aperture of the second pair of tether apertures, and the intermediate portion of the tether extends between the first and second end portions of the tether.

14. The system of claim 13, wherein the second bone anchor comprises a head portion and a shaft portion extending from the head portion, wherein the second pair of tether apertures are formed by a longitudinal opening in the head portion and a lateral through hole through the shaft portion of the second anchor that is in communication with the longitudinal opening.

15. The system of claim 14, wherein the first end portion of the tether extends through the first tether aperture from the head portion to the shaft portion, and the second end portion of the tether extends through the second tether aperture from the shaft portion to the head portion.

16. The system of claim 13, wherein the first and second end portions of the tether are arranged in a slip knot that is configured to retain the length of the intermediate portion of the tether extending between the first and second anchors.

17. The system of claim 1, wherein the first bone anchor is longitudinally elongated, and wherein the first bone anchor is longitudinally oriented with respect to the longitudinally elongated cavity of the insertion tube, and at least a portion of the first bone anchor along the longitudinal length thereof is removably received within a portion of the longitudinally elongated cavity of the insertion tube at the tip thereof.

18. The system of claim 1, wherein the instrument further comprises a tether retainer comprising a pair of projections with wing portions and slot between the wing portions, and wherein end portions of the tether wrap about the projections beneath the wing portions and pass through the slot at least once.

19. The system of claim 18, wherein the instrument further comprises a tether retainer keeper comprising an elastic member with an opening, the tether retainer keeper being configured to extend about the projections beneath the wing portions and over the end portions of the tether wrapped thereabout in a resiliently deformed expanded state.

20. The system of claim 1, wherein the second bone anchor comprises a head portion and a shaft portion extending from the head portion, wherein the implant further comprises a washer member with a through hole, the washer member being configured to engage the head portion of the second bone anchor with the shaft portion of the second bone anchor extending through the through hole, and wherein the washer member is mounted on the instrument with the insertion tube and the tether extending through the through hole.

21. A method of stabilizing first and second bones, comprising:
obtaining an implant system comprising a stabilization implant mounted on an implantation instrument, wherein:
the implant comprises first and second bone anchors and a flexible tether coupling the first and second bone anchors;
the instrument comprises a handle portion, an adjustment member, a longitudinally elongated insertion tube extending from the handle portion to a tip and comprising a longitudinally elongated cavity, and a deployment portion coupled with the adjustment member and threadably engaged with a portion of the instrument,
the deployment portion comprises a base portion extending within the handle portion and a longitudinally elongated deployment rod portion slidably received within the cavity of the insertion tube, and
wherein at least a portion of the first bone is removably received within the cavity of the insertion tube at the tip thereof;
passing the first bone anchor and a portion of the insertion tube from an outer side of the first bone through a portion of the first and second bones such that the second bone anchor is positioned past an outer side of the first bone and the first bone anchor is positioned adjacent to an outer side of the second bone;
manually adjusting the adjustment member in a first direction to threadably drive the deployment rod portion longitudinally within the cavity of the insertion tube toward the tip thereof to force the first bone anchor out from the cavity;
adjusting the location of the second bone anchor along the tether to force to shorten a length of the tether between the first and second bone anchors to position the second bone anchor against the outer side of the first bone; and
tying end portions of the tether extending from the second bone anchor into a surgical knot to fix the arrangement of the implant and stabilize the first and second bones.

22. The method of claim 21, wherein the first and second bones are bones of a syndesmosis joint.

23. An instrument for implanting an implant through first and second bones, comprising:
a handle portion;
a manually engageable adjustment member movably coupled with the handle portion;
a longitudinally elongated insertion tube extending from a front portion of the handle portion to a tip and comprising a longitudinally elongated cavity, the cavity at the tip being configured to removably house at least a portion of a first bone anchor of the implant; and
a deployment portion coupled with the adjustment member and threadably engaged with a portion of the instrument, the deployment portion comprising a base portion extending within the handle portion and a longitudinally elongated deployment rod portion slidably received within the cavity of the insertion tube, and
wherein movement of the adjustment member in a first direction adjusts the threaded engagement of the deployment portion with respect to the instrument to threadably drive the deployment rod portion longitudinally within the cavity of the insertion tube toward the tip thereof.

24. The instrument of claim 23, wherein the deployment portion is threadably engaged with the portion of the instrument via the base portion being threadably engaged with an inner portion of the handle portion.

25. The instrument of claim 23, wherein the handle portion further comprises a bone anchor retention portion configured to removably retain a second bone anchor of the implant that is coupled to the first bone anchor via a flexible tether.
